(19) United States
(12) Patent Application Publication
(10) Pub. No.: US 2011/0206781 A1
(43) Pub. Date: Aug. 25, 2011

(54) METHOD TO MODULATE HEMATOPOIETIC STEM CELL GROWTH

(76) Inventors: Leonard I. Zon, Wellesley, MA (US); Trista E. North, Newton Center, MA (US); Wolfram Goessling, West Roxbury, MA (US)

(21) Appl. No.: 12/994,527

(22) PCT Filed: May 28, 2009
(86) PCT No.: PCT/US09/45442

§ 371 Date: May 12, 2011

Related U.S. Application Data
(60) Provisional application No. 61/056,621, filed on May 28, 2008.
(60) Provisional application No. 61/177,720, filed on May 13, 2009.

(51) Int. Cl. A61K 33/00; C12N 5/0789; A61K 31/197; A61K 31/517; A61K 31/138; A61K 31/4418; A61K 31/7048; A61K 31/198; A61K 31/295; A61K 31/165; A61K 31/4985; A61K 31/137; A61K 31/44; A61K 31/353; A61K 31/216; A61P 7/00

(52) U.S. Cl. 424/718; 435/377; 514/562; 514/252.17; 514/652; 514/356; 514/26; 514/565; 514/502; 514/620; 514/250; 514/653; 514/352; 514/456; 514/654; 514/532; 514/655

(57) ABSTRACT

Described herein are methods, compositions and kits related to manipulating hematopoietic stem cells (HSC) and more particularly to methods, compositions and kits related to increasing the number of hematopoietic stem cells in vitro, ex vivo and/or in vivo. Also described are methods, compositions and kits related to making an expanded population of HSC and methods, compositions and kits related to using the expanded population of HSC. For example, HSC growth may be enhanced by contacting the nascent stem cells or HSC with an agent that stimulates the nitric oxide signaling pathway.

### CROSS REFERENCE TO RELATED APPLICATIONS

This application claims the benefit of U.S. Provisional Patent Application No. 61/056,621, filed May 28, 2008, and U.S. Provisional Patent Application No. 61/177,720, filed May 13, 2009, which applications are incorporated herein by reference in their entirety.

### GOVERNMENT SUPPORT

This invention was made with government support under Grant No. CA103846-02, awarded by the National Institutes of Health. The U.S. government of the has certain rights in the invention.

### FIELD OF THE INVENTION

This invention relates to hematopoietic stem cells, and more particularly to methods, kits and compositions for manipulating hematopoietic stem cells. The present embodiments provide for modulators that either increase or decrease hematopoeitic stem cell (HSC) populations. More specifically, for example, modulators of nitric oxide synthesis and signaling affect HSC growth.

### BACKGROUND

Stem cell research holds extraordinary potential for the development of therapies that may change the future for those suffering from diseases such as leukemia, diabetes, and anemia. Much research focuses on the exploration of stem cell biology as a key to treatments for diseases. Through an understanding of the role of stem cells in normal development, researchers seek to capture and direct the innate capabilities of stem cells to treat many conditions. Research is on-going in a number of areas simultaneously: examining the genetic and molecular triggers that drive embryonic stem cells to develop in various tissues; learning how to push those cells to divide and form specialized tissues; culturing embryonic stem cells and developing new lines to work with; searching for ways to eliminate or control Graft vs. Host Disease by eliminating the need for donors; and generating a line of universally transplantable cells.

Hematopoietic stem cells (HSCs) are derived during embryogenesis in distinct regions where specific inductive events convert mesoderm to blood stem cells and progenitors. There remains a need to elucidate the relationships between particular biomolecules, chemical agents, and other factors in these inductive events. For example, there remains a need to identify which biomolecules or chemical agents show promise in manipulating the HSC population for a desired purpose, such as increasing a HSC population for research or therapeutics.

### SUMMARY

Described herein are methods, compositions and kits related to manipulating stem cells and more particularly to methods, compositions and kits related to increasing the number of hematopoietic stem cells in vitro, ex vivo, and in vivo. Also described are methods, compositions and kits related to making an expanded population of hematopoietic stem cells (HSCs) and methods, compositions and kits. The compositions and methods of the present embodiments provide for HSC modulators, which are agents that increase HSC numbers as desired by a particular indication. In particular, for example, the present invention provides for nitric oxide (NO) signaling as a conserved regulator of HSC development in vitro, ex vivo, or in vivo. Moreover, according to the methods for the present invention, modulation of blood flow and/or NO signaling may be therapeutically beneficial for patients undergoing, for example, stem cell transplantation.

During vertebrate embryogenesis, hematopoietic stem cells (HSCs) arise in the aorta-gonads-mesonephros (AGM) region. Blood flow is a conserved regulator of HSC formation. In Zebrafish, chemical blood flow modulators regulated HSC development, and silent heart (sih) embryos, lacking a heartbeat and blood circulation, exhibited severely reduced HSCs. Flow-modifying compounds primarily affected HSC induction after the onset of heartbeat; however, nitric oxide (NO) donors regulated HSC number even when treatment occurred before the initiation of circulation, and rescued HSCs in sih mutants. Morpholino knockdown of nos1 (nnos/enos) blocked HSC development, and its requirement was shown to be cell autonomous. In the mouse, Nos3 (eNos) was expressed in HSCs in the AGM. Intrauterine Nos inhibition or embryonic Nos3 deficiency resulted in a reduction of hematopoietic clusters and transplantable murine HSCs. The present invention thus links blood flow to AGM hematopoiesis and identifies NO as a conserved downstream regulator of HSC development: circulation functions to provide inductive signals to specific regions of the embryonic vasculature, making it competent to produce HSCs de novo.

An embodiment of the present invention provide for modulators of NO synthesis and NO signaling that affect HSCs. For example, NO pathway modulators (and associated downstream pathway modulators) may be used for the induction of HSCs from a stem cell population including embryonic stem cell (ESC), induced pluripotent stem cell (iPSC or iPS), or AGM HSC populations.

Another embodiment of the present invention provide for modulators of NO synthesis and NO signaling that affect HSCs. For example, NO pathway modulators (and associated downstream pathway modulators) may be used for promoting hematopoietic stem cell growth in a subject, by administering at least one HSC modulator and a pharmaceutically acceptable carrier.

One embodiment of the invention provides for modulators that increase HSCs, such as the α1-adrenergic blocker Doxasozin; the β1-adrenergic blocker Metoprolol; the Ca²⁺-channel blocker Nifedipine; the cardiac glycoside Digoxin, a modulator of Na⁺/K⁺; the NO donor S-nitroso-N-acetyl-penicillamine (SNAP); L-ARG; Todralazine; Sodium Nitroprus side; Atenolol; Pronethalol; Pindolol; Fendiline; Nicardipine; Strophanthidin; Lanatoside; Peruvoside; Histamine; Hydralazine; Todralazine; Nitrosothiols; Diazetine dioxides; Sydnonimines; N-Nitrosamines; Oximes; Nitroimines; C-nitroso compounds; Fluoroxans and benzofuroxans; Oxatriazole-5-imines; Organic nitrates; Organic nitrites; Metal-NO complexes; N-Nitrosamines; N-Hydroxynitrosamines; Hydroxylamines; N-Hydroxyguanidienes; Hydroxyureas; GTN; GNSO; SIN-1; Angell's Salt; DEA/NO; PAPA/NO; SPER/NO; PROLI/NO; MAMA/NO; DETA/NO; NO-Aspirin; NO-Indomethacin; NO-Ibuprophen; NO-Salicylic Acid; and NO-Sulindac.

In an aspect of the invention, the modulators NO and SNAP increase HSC populations in the absence of circulation, hence, another aspect of the invention provides for a method for promoting HSC growth by contacting a nascent stem cell population (e.g., ES, iPSC, or AGM HSC) with NO donors or NO signaling pathway agonists. In another aspect, the nascent stem cell population may be collected from peripheral blood, cord blood, chorionic villi, amniotic fluid, placental blood, or bone marrow.

Another embodiment of the present invention provides a method for promoting HSC expansion ex vivo, comprising incubating a nascent stem cell population or HSC population in the presence of at least one HSC modulator, such as NO or SNAP. Another embodiment of the present invention provides a method for promoting HSC expansion ex vivo, comprising collecting HSC source sample (e.g., peripheral blood, cord blood, amniotic fluid, placental blood, bone marrow, chorionic villi) and storing it in the presence of at least one HSC modulator such as NO and/or SNAP. A particular embodiment provides for a kit comprising a container suitable for HSC-source sample storage in which the container is pre-loaded with at least one HSC modulator that increases HSCs. An additional embodiment provides a kit comprising a container suitable for HSC-source sample storage and a vial containing a suitable amount of at least one HSC modulator that increases HSCs. A further embodiment of the present invention provides a method for promoting HSC expansion ex vivo, in which the nascent HSC source is contacted with NO and or SNAP, or a derivatives thereof, at initial collection, during processing, at storage, upon thawing, or during transfusion.

Another embodiment of the invention provides for modulators that inhibit HSCs, such as the α-agonist Ergotamine; the β1-agonist Epinephrine; BayK8644; the Nos inhibitor N-nitro-L-arginine methyl ester (L-NAME); Chrysin; the angiotensin-converting enzyme (ACE) inhibitor Enalapril; Ephedrine; Methoxamine; Mephentermine; Propranolol; Nerifolin; Proadifen; Ambroxol; or Captopril. In a particular embodiment, the HSC modulator is one or more of the compounds selected from the group consisting of Ergotamine, Epinephrine, BayK8644, L-NAME, Chrysin, Enalapril, Ephedrine, Methoxamine, Mephentermine, Propranolol, Nerifolin, Proadifen, Ambroxol, and Captopril.

Another embodiment of the present invention provides for HSC modulators that exert an effect during active circulation (i.e., after heart beat is initiated) such as Doxazosin, Propanolol, Metopolol, Nifedipine, Digoxin, SNAP, Bradykinin and Trodralazine, which increase HSCs; and Dihydroergotamine, epinephrine, BayK8644, L-NAME and Enalapril, which decrease HSCs.

In general, the compounds of the present embodiments can be applied ex vivo to cells or organ tissue (e.g., bone marrow tissue). Alternatively, the modulators may be used to enhance or inhibit in vitro HSC populations. Additionally, the compounds of the present embodiments can be applied systemically to the patient, or in a targeted fashion to the organ in question (e.g., bone marrow).

### DESCRIPTION OF THE DRAWINGS

FIG. 1 demonstrates that the modulation of vascular flow affects HSC formation in Zebrafish. FIGS. 1A-1M reflect the effect of blood flow modifiers on runx1/cmyb+HSC formation. Zebrafish were exposed to chemicals (10 mM) from 10 somites-36 hpf and subjected to runx1/cmyb in situ hybridization. Photomicrographs were taken with Nomarski optics at 40× magnification. Representative examples from after drug treatment are shown. FIG. 1L is the effect of todralazine (10 μM; 67 inc/84); FIG. 1M the effect of drug treatment on runx1 expression, quantified by qPCR; FIG. 1N the effect of drug treatment on the diameter of the dorsal aorta in vivo. Transgenic fli:GFP fish were treated with chemicals and imaged by confocal microscopy at 36 hpf; all treatments were statistically significant from the control (mean±SD, ANOVA, p<0.001). Color versions of FIGS. 1-22 are available in North et al., 137 Cell 1-13, Suppl. (May 15, 2009).

FIG. 2 demonstrates that a beating heart is required for HSC formation and artery development. FIGS. 2A-2H show the effect of sih mutation on HSC and vascular formation at 36 hpf. FIGS. 2A and 2E show runx1/cmyb expression is greatly reduced in sih^{-/-} embryos compared to WT siblings. FIGS. 2B and 2F show flk1 expression reveals a grossly normal vascular pattern in sih^{-/-} embryos; changes in the development of the intersomitic vessels and vascular plexus were noted in some animals. FIGS. 2C and 2F show ephB2 expression is diminished in sih^{-/-} embryos. FIGS. 2D and 2H show flt4 expression is expanded in sih^{-/-} embryos. FIG. 2I shows expression levels of HSC (runx1, cmyb), vascular (flk), and arterial (ephB2) markers are significantly decreased in sih^{-/-} embryos compared to sibling controls (mean±SD, t test, p<0.05, n=3), as measured by qPCR at 36 hpf. FIGS. 2J and 2K show the sih mutation has no effect on primitive hematopoiesis as seen by benzidine staining at 36 hpf; in the absence of a heartbeat blood is pooled in the major vessels.

FIG. 3 illustrates how NO signaling, prior to the onset of cardiac activity, can affect HSC formation. FIGS. 3A-G show the effect of vasoactive drugs (10 mM) on HSC formation before and after the onset of heartbeat at 24 hpf, after exposure to chemicals from either 10 somites-23 hpf or from 26-36 hpf. FIG. 3A shows most vasoactive drugs do not affect HSC formation when applied prior to the onset of heartbeat, while NO modifiers influenced HSC development even prior to heart beat initiation. The percentage of embryos (n>20) with altered runx1/cmyb expression is indicated. FIGS. 3B-3G are representative examples of flow-modifying drugs on runx1/cmyb expression. FIGS. 3H-3P show the specificity of NO signaling in HSC formation. NO donors enhanced and diminished HSCs; inactive D-enantiomers had no effect. FIGS. 3Q-3S show the effects of NO modulation on HSC number by in vivo confocal imaging in cmyb:GFP; lmo2:dsRed transgenic embryos. FIGS. 3T-3Y show the effects of downstream modifiers of NO signaling on runx1/cmyb expression. FIGS. 3U and 3X show inhibition of soluble guanyl cyclase by ODQ (10 μM) decreases runx1/cmyb expression in WT and SNAP treated embryos. FIGS. 3V and 3Y shows that inhibition of PDE V by MBMQ (10 μM) increases HSC formation in WT embryos and further enhances the effects of SNAP.

FIG. 4 demonstrates that NO signaling affects Zebrafish HSC formation independent of heartbeat. FIGS. 4A-4I show WT and sih^{-/-} mutants were exposed to DMSO and SNAP (10 μM) from 10 somites-36 hpf. FIGS. 4A-4D show in situ hybridization for runx1/cmyb. SNAP rescues HSC formation in sih^{-/-} mutants. FIG. 4B shows runx1/cmyb+ cells highlighted by arrowheads. FIG. 4E shows qPCR for runx1. * statistically significant versus the WT, mean±SD, ANOVA, p<0.001, n=5. FIGS. 4F-4I show the effect of heartbeat and SNAP on ephrinB2 expression, highlighted by arrowheads. FIGS. 4J-4U show the effect of L-NAME on HSC formation in embryos concurrently treated with blood flow-modifying agents. L-NAME inhibits the effects of doxazosin ([FIG. 4M], 7 inc/36 observed), metoprolol ([FIG. 4O], 3 inc/31) and nifedipine ([FIG. 4Q], 4 inc/28), but not of digoxin ([FIG. 4S], 16 inc/29) and todralazine ([FIG. 4U], 20 inc/33).

FIG. 5 illustrates that nos1 is required for HSC formation in zebrafish. FIGS. 5A-5H show in situ hybridization for runx1/cmyb at 36 hpf. FIGS. 5C and 5E show nos1 knockdown (40 μM) decreased HSC formation. FIGS. 5 D and 5F show MO (ATG and splice site) against nos2 (40 μM) had no effect on HSC development. FIGS. 5B, 5G, and 5H show chemical nos inhibition confirmed the specific requirement for nos1: embryos exposed to nonspecific (L-NAME; 10 μM) and nos1-selective (S-methyl-L-thiocitrulline; 10 mM) inhibitors demonstrated decreased HSC formation; nos2-selective inhibition (1400W; 10 μM) had minimal impact. FIG. 5I shows WT and sih^{-/-} embryo extracts (n=20) were subjected to qPCR (mean±SD; * nos1, WT versus sih, t test, p<0.001, n=3; nos2, WT versus sih, p=0.385, n=3). FIGS. 5J and 5K show effect of flow-modifying chemicals (10 μM, 10 somites-36 hpf) on nos1 and nos2 expression; nos1 is significantly regulated by most compounds tested. Mean±SD; * significant versus control, ANOVA, p<0.01, n=3.

FIG. 6 shows that the effect of NO signaling on HSC development is cell autonomous. FIG. 6A shows cells from cmyb: GFP transgenic donor embryos, injected with nos1 ATG MO or control MO, were transplanted into lmo2:dsRed recipients at the blastula stage. FIG. 6B shows donor contribution to HSC formation assessed by confocal microscopy at 36 hpf. Shown are the merged picture on the top, merge in the middle, and a high-magnification view of fluorescence only on the bottom. cmyb: GFP donor-derived HSCs in recipients are highlighted by arrowheads. FIG. 6C shows nos1 MO donors never contributed to HSC formation; the presence of cmyb: GFP-derived donor cells in the eye is indicative of a successful transplant. FIG. 6D shows HSC chimerism in transplanted embryos (control versus nos1 MO, Fisher's exact test, p=0.0065, n>8).

FIG. 7 illustrates that the effect of NO signaling on HSC development in the AGM is conserved in mice. FIGS. 7A-7H are FACS analysis of dissociated AGM cells in WT and Nos KO mice at e11.5. Nos3^{-/-} mice exhibited a decrease in the Sca1/cKit⁺ and CD45/VE-Cadherin⁺ populations, while deletion of Nos1 had no significant effects. FIGS. 7I-7L show histological sections through the AGM region of e11.5 embryos; the inset represents a high-magnification view around the hematopoietic clusters. L-NAME exposure causes absence of hematopoietic clusters; Nos3^{-/-} mice exhibit smaller cluster size, while Nos1^{-/-} does not impair cluster formation. Serial sections through the entire aorta of at least ten embryos per genotype/treatment were analyzed. FIGS. 7M and 7N show the effect of NO signaling on AGM HSC function. AGM regions of somite stage-matched WT, L-NAME treated or Nos3^{-/-} progeny were subdissected at e11.5 and transplanted into sublethally irradiated recipients. L-NAME exposure or Nos3 deletion embryos significantly reduced CFU-S12 spleen colony formation (mean±SD; * sig versus control; p<0.001; ** sig versus L-NAME; p<0.05; ANOVA, n≧5) (FIG. 7M). Diminished NO signaling significantly decreased embryonic donor cell chimerism rates in individual recipient mice at 6 weeks after transplant (* sig versus control, p<0.05, ANOVA, n≧5) (FIG. 7N).

FIG. 8 shows that modulation of blood flow affects HSC formation in zebrafish. Zebrafish were exposed to chemicals (10 μM) from 10 somites to 36 hpf and subjected to in situ hybridization for runx1/cmyb. FIG. 8A is a summary of the effects of drugs included in several chemical screen libraries and their mechanism of action. FIG. 8B shows stage-specific regulation of genes involved in blood flow regulation in the hematopoietic and endothelial compartments of the developing zebrafish embryo. Cell populations were isolated by FACS in transgenic Zebrafish embryos and subjected to microarray analysis. nos1 is upregulated in the HSC compartment at 36 hpf.

FIG. 9 illustrate that nitric oxide mediates the effect of blood flow on HSCs. FIGS. 9A-9I show the effect of chemical modifiers of blood flow on vascular diameter in vivo. Transgenic fli:GFP fish were treated with chemicals (10 μM) from 10 somites to 36 hpf and imaged by confocal microscopy. Microscopy images with measurement of the diameter of the dorsal aorta in representative samples of drug-treated embryos. The inset shows a lower magnification image to visualize the entire tail region. The red bars indicate the width of the dorsal aorta.

FIG. 10 shows that the silent heart mutation does not affect primitive hematopoiesis or mesodermal and endodermal development at 36 hpf. FIGS. 103A-10D show in situ hybridization (n>25 per treatment) for globin and myeloperoxidase (mpo) demonstrates pooling of blood cells due to the absent heartbeat, but no quantitative changes for primitive erythropoiesis or myelopoiesis in sih mutants. FIGS. 10E and 10F show somite formation as depicted by in situ hybridization for myosin heavy chain (mhc) is normal in sih mutants, indicating that other mesodermal organs develop normally. FIGS. 10G, 10H show Endoderm development, visualized by foxa3 expression, is not affected in sih mutants.

FIG. 11 shows that the modulation of NO has dose-dependent effects on HSC formation. FIGS. 11A-11L show embryos (n>25 per treatment) were exposed to increasing doses of L-NAME (B-F) or SNAP (H-L) from 10 somites to 36 hpf. With increasing L-NAME dose, HSC formation was progressively diminished. Similarly, SNAP lead to a dose-dependent increase of runx1/cmyb expression. Doses >20 μM for each drug led to gross morphological abnormalities.

FIG. 12 shows NO modulation does not affect primitive hematopoiesis or development of mesodermal and endodermal structures. FIGS. 12A-12R show embryos (n>25 per treatment) were exposed to control, L-NAME or SNAP from 10 somites to 36 hpf. FIGS. 12A-12F show expression of the vascular marker flk1 is minimally altered by NO modulation. FIGS. 12G-12R show primitive erythro-(globin) and myelopoiesis (mpo) as well as early muscle (mhc) and endoderm (foxa3) development are not affected by changes in NO signaling. FIG. 12S is the quantitation of HSC number in confocal microscopy analysis of cmyb:GFP; lmo2:dsRed embryos (FIG. 3Q-3S) reveals significant changes in response to NO modulation (* sig vs. control, ANOVA, p<0.001, n=5).

FIG. 13 reflects a time course analysis that reveals time-specific effect of NO modulation on HSCs. FIGS. 13A-130 shows embryos were exposed to L-NAME and SNAP from 10 somites until fixation (22-72 hpf). FIGS. 13A-16F shows that SNAP exposure does not increase the expression of HSC markers at early stages. FIGS. 12G-120 show that nos inhibition by L-NAME does not cause a delay in HSC development that can be compensated for at later developmental stages.

FIG. 14 shows that Nos inhibition increases apoptosis within the AGM. FIGS. 14A-14D show Zebrafish embryos were exposed to L-NAME and SNAP from 10 somites to 36 hpf and processed for TUNEL staining. L-NAME treatment significantly enhanced the number of apoptotic cells in the zebrafish AGM tail region. * sig vs. control, p<0.001, ANOVA, n=10.

FIG. 15 shows that NO signaling affects vascular and HSC development. FIG. 15A shows qPCR for ephrinB2 in WT and sih^{-/-} in the presence and absence of SNAP. * sig vs. control, p<0.05, ANOVA, n=5. FIGS. 15B-15E show the effects of bradykinin (10 μM) on runx1/cmyb expression in wild-type and sih^{-/-} embryos at 36 hpf. runx1/cmyb positive cells are highlighted by arrowheads.

FIG. 16 demonstrates the effect of nos1 MO inhibition is dose-dependent. FIG. 16A RT-PCR performed on cohorts of twenty pooled nos1 splice site MO (40 μM) and control MO injected embryos. The control injected embryos exhibited the expected fragment length (300 bp), while the PCR product after splice site MO injection is shorter as expected. Actin is shown as a control. FIG. 16B-16E shows that increasing nos1 knockdown by increasing doses of MO caused progressive decrease in runx1/cmyb expression. FIGS. 16F-16I nos2 knockdown did not affect HSC formation. FIGS. 16J-16L show immunoreactivity to both anti-mouse Nos1 and Nos3 antibody was present in zebrafish embryos at 36 hpf. Nos3 reactivity was found in the vasculature, neural tube and endodermal tissues.

FIG. 17 relates to blastula transplant controls. FIG. 17A shows uninjected control cmyb:GFP embryo; FIG. 17B is uninjected control lmo2:dsRed embryo; FIG. 17C is recipient cmyb:GFP embryos injected with nos1 MO had a grossly normal phenotype normal and express cmyb:GFP robustly in the eye, and neural crest; greatly reduced expression was found in the HSC compartment. Donor-derived endothelial cells could be seen in red fluorescence.

FIG. 18 evidences that NO modifies the effects of notch signaling on HSC formation. Zebrafish embryos were assessed by in situ hybridization for runx1/cmyb at 36 hpf. FIGS. 18A-18D show wild-type and mib^{-/-} mutant embryos were exposed to DMSO and SNAP (10 μM) from 10 somites to 36 hpf. NO rescued the HSC defect in mib^{-/-} embryos. FIGS. 18E-S11H show inhibition of NO by L-NAME (10 μM) diminished the enhancing effect of constitutive notch activation in NICD transgenic zebrafish embryos.

FIG. 19 demonstrates that NICD-mediated elevation of ephB2 is blocked by nos inhibition. Zebrafish embryos were assessed by in situ hybridization for ephB2 at 36 hpf (n>25/condition). FIGS. 19A-19D shoe the effect of L-NAME on ephB2 expression in WT and NICD transgenic embryos; L-NAME treatment blocked the notch-mediated increase in ephB2 staining.

FIG. 20 shows that NO modifies the effects of wnt signaling on HSC formation. FIGS. 513A-513H show that inducible wnt pathway transgenic embryos were subjected to heatshock at 38° C. for 20 mins at 10 somites and then exposed to chemicals (10 μM) until 36 hpf and subjected to runx1/cmyb in situ hybridization. FIGS. 20A-20D show dkk1 induction diminished HSC number, which can be rescued by SNAP. FIGS. 20E-20H show L-NAME inhibited the wnt8-mediated enhancement of HSCs.

FIG. 21 demonstrates Nos3 expression characterizes the transplantable HSC population in the AGM. FIGS. 21A-21D show DIC and fluorescence microscopy of sections through the aorta of Nos3:GFP transgenic (FIGS. 21A, 21B) and wild-type control (FIGS. 21C, 21D) mouse embryos at e8.5. Individual cell nuclei are indicated by DAPI staining. Hematopoietic clusters are highlighted by a box. The arrow indicates a subaortic patch of HSCs. The arrowhead indicates the lack of GFP fluorescence within erythrocytes in the lumen of the vessel.

FIG. 21E is a representative flow cytometric analysis of E11.5 Nos3:GFP transgenic AGM cells. Single cell suspensions, gated for live (Hoechst negative) mononuclear cells (SSC; FSC), were analyzed for HSC marker expression. The top right panel shows fractionation of AGM cells fall into Nos3:GFP negative, medium and high expression groups. The bottom right panel shows a histogram plot of GFP expression in Nos3:GFP transgenic (black outlined curve) and wild type (grey curve) E11.5 AGM cells gated on viable, mononuclear C-kit^{hi}CD45^{med}CD34^{med}VE-cadherin^{med} cells, of which 9.4±8.9% were negative for Nos3:GFP expression, 90.5±8.2% expressed Nos3:GFP to an intermediate level and no cells exhibited high levels of Nos3:GFP. Three independent experiments were performed using a total of 40 Nos3:GFP transgenic embryos and 25 wild-type embryos.

FIG. 21F shows Nos3:GFP^{lo} expressing AGM cells contain the transplantable population. Suspension of AGM cells were sorted into Nos3:GFP negative, intermediate, and high fractions. Donor-derived cells in recipient peripheral blood at four-months post-transplantation were detected by PCR, with >10% donors marked cells considered positive.

FIG. 22, inhibition of NO signaling decreases phenotypic and functional. FIGS. 22A-22G summarize FACS analysis of subdissected AGM at E11.5. FIG. 22A is a representative control FACS plots showing (top to bottom) an unstained AGM cell suspension; a FL1⁺ (VE-cadherin), FL2 isotype control; a FL2+ (CD45); FL1 isotype control. FIGS. 22C-22F show the CD45⁺/VE-Cad⁺ and sca1⁺/ckit⁺ cell populations are significantly diminished in L-NAME and Nos3^{-/-} embryos. * sig vs. control, p<0.05, ANOVA, n 8. FIGS. 22D and 22G show that the VE-Cad⁺ and c-Kit⁺ populations were significantly decreased in L-NAME-treated embryos. * sig vs. control, p<0.05, ANOVA, n≧8. FIGS. 22H, 22I show histological analysis of Runx1:lacZ mice revealed lack of hematopoietic clusters and reduced Runx1⁺ cells in the AGM of embryos from L-NAME treated females. Serial sections through the entire aorta of 10 embryos per genotype/treatment were analyzed. FIG. 22J relates to L-NAME treatment of pregnant females decreased functional embryonic progenitors, as measured in spleen colony formation on day 8 post-AGM transplantation into irradiated mice. * sig vs. control, p<0.001, t-test, n≧10. FIGS. 22K-226L shows diminished NO signaling in the AGM of either L-NAME exposed or Nos3^{-/-} e11.5 embryos caused a decrease in transplantable HSCs, assessed both by average chimerism or engraftment >1% at six weeks post transplantation. * sig vs. controls, p<0.05, ANOVA, n≧5.

### DETAILED DESCRIPTION

It should be understood that this invention is not limited to the particular methodology, protocols, and reagents, etc., described herein and as such may vary. The terminology used herein is for the purpose of describing particular embodiments only. Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Hematopoietic stem cells (HSC) are primitive cells capable of regenerating all blood cells. During development, hematopoiesis translocates from the fetal liver to the bone marrow, which then remains the site of hematopoiesis throughout adulthood. Once hematopoiesis has been established in the bone marrow, the hematopoietic stem cells are not distributed randomly throughout the bone cavity. Instead, the hematopoietic stem cells are found in close proximity to the endosteal surfaces. The more mature stem cells (as measured by their CFU-C activity) increase in number as the distance from the bone surface increases. Finally, as the central longitudinal axis of the bone is approached terminal differentiation of mature cells occurs. Given the relationship between the hematopoietic stem cells and the endosteal surfaces of the bone, the osteoblast may play a role in hematopoiesis. Osteoblastic cells, for example, support the growth of primitive hematopoietic cells through the release of G-CSF and other growth factors.

Expanding the number of bone marrow derived stem cells is useful in transplantation and other therapies for hematologic and oncologic disease. As described in the methods herein, HSC numbers are increased in vitro, ex vivo, or in vivo. A method of increasing stem cell numbers reduces the time and discomfort associated with bone marrow/peripheral stem cell harvesting and increases the pool of stem cell donors. Currently, approximately 25% of autologous donor transplants are prohibited for lack of sufficient stem cells. In addition, less than 25% of patients in need of allogeneic transplant can find a histocompatible donor. Umbilical cord blood banks currently exist and cover the broad racial make-up of the general population, but these banks are currently restricted to use in children due to inadequate stem cell numbers in the specimens for adult recipients. A method to increase stem cell numbers permits cord blood to be useful for adult patients, thereby expanding the use of allogeneic transplantation.

Methods for making an expanded population of HSC are provided comprising administering a modulator such as an agonist of the NO signaling pathway to an unexpanded population of HSC or to a mixture of HSC and HSC-supporting cells under conditions that allow the unexpanded population of HSC to increase in number to form an expanded population of HSC. As used herein, an expanded population of HSC refers to a population of HSC comprising at least one more HSC, 10% more, 20% more, 30% more or greater as compared to the number of HSC prior to or in the substantial absence of administration of the NO signaling agonist in a control population. An unexpanded population of HSCs refers to an HSC population prior to or in the substantial absence of exposure to an exogenous NO signaling agonist. An unexpanded population of HSC and HSC supporting cells refers to an HSC population and HSC supporting cells prior to or in the substantial absence of exposure to an exogenous NO signaling agonist. Thus, a method for increasing the number of HSC in a subject comprising administering a NO signaling agonist to the subject is also described. The HSCs are obtained from any subject and thus, are autologous or heterologous donor material. Optionally, the stem cells are human stem cells. The HSC are obtained from any subject and thus, are autologous or heterologous donor material. Optionally, the stem cells are human stem cells.

The expanded population of stem cells are harvested, for example, from a bone marrow sample of a subject or from a culture. Harvesting hematopoietic stem cells is defined as the dislodging or separation of cells. This is accomplished using a number of methods, such as enzymatic, non-enzymatic, centrifugal, electrical, or size-based methods, or preferably, by flushing the cells using culture media (e.g., media in which cells are incubated) or buffered solution. The cells are optionally collected, separated, and further expanded generating even larger populations of HSC and differentiated progeny.

As described herein, the expanded population of HSC comprise short term HSC (ST-HSC) or long term HSC (LT-HSC). Thus, provided are methods of providing an expanded population of hematopoietic stem cells to a subject comprising administering to the subject the expanded population of hematopoietic stem cells described herein or made by the methods described herein. Thus, methods for making an expanded population of hematopoietic stem cells comprise administering an agent that enhances NO signaling to an unexpanded population of HSC or a mixture of HSC and HSC-supporting cells under conditions that allow the unexpanded population of HSC to increase in number to form an expanded population of HSC. The expanded population of HSC are optionally used to make blood cells. Thus, methods are provided for making blood cells comprising differentiating hematopoietic stem cells into blood cells, wherein the HSC are derived from the expanded population of HSC as described or according to the methods as described herein. The blood cells are optionally administered to a subject in need. Optionally, the subject is the same subject from which the unexpanded population of HSC or mixture of HSC and HSC-supporting cells was derived.

HSC as used herein refer to immature blood cells having the capacity to self-renew and to differentiate into more mature blood cells comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages). Hematopoietic stem cells are interchangeably described as stem cells throughout the specification. It is known in the art that such cells may or may not include CD34+ cells. CD34+ cells are immature cells that express the CD34 cell surface marker. CD34+ cells are believed to include a subpopulation of cells with the stem cell properties defined above. It is well known in the art that hematopoietic stem cells include pluripotent stem cells, multipotent stem cells (e.g., a lymphoid stem cell), and/or stem cells committed to specific hematopoietic lineages. The stem cells committed to specific hematopoietic lineages may be of T cell lineage, B cell lineage, dendritic cell lineage, Langerhans cell lineage and/or lymphoid tissue-specific macrophage cell lineage. In addition, HSCs also refer to long term HSC (LT-HSC) and short term HSC (ST-HSC). A long term stem cell typically includes the long term (more than three months) contribution to multilineage engraftment after transplantation. A short term stem cell is typically anything that lasts shorter than three months, and/or that is not multilineage. LT-HSC and ST-HSC are differentiated, for example, based on their cell surface marker expression. LT-HSC are CD34−, SCA-1+, Thy1.1+/lo, C-kit+, Un−, CD135−, Slamfl/CD150+, whereas ST-HSC are CD34+, SCA-1+, Thy1.1+/lo, C-kit+, lin−, CD135−, Slamfl/CD150+, Mac-1 (CD1Ib)lo (“lo” refers to low expression). In addition, ST-HSC are less quiescent (i.e., more active) and more proliferative than LT-HSC. LT-HSC have unlimited self renewal (i.e., they survive throughout adulthood), whereas ST-HSC have limited self renewal (i.e., they survive for only a limited period of time). Any of these HSCs can be used in any of the methods described herein.

HSC are optionally obtained from blood products. A blood product includes a product obtained from the body or an organ of the body containing cells of hematopoietic origin. Such sources include unfractionated bone marrow, umbilical cord, peripheral blood, liver, thymus, lymph and spleen. All of the aforementioned crude or unfractionated blood products can be enriched for cells having hematopoietic stem cell characteristics in a number of ways. For example, the more mature, differentiated cells are selected against, via cell surface molecules they express. Optionally, the blood product is fractionated by selecting for CD34+ cells. CD34+ cells include a subpopulation of cells capable of self-renewal and pluripotentiality. Such selection is accomplished using, for example, commercially available magnetic anti-CD34 beads (Dynal, Lake Success, N.Y.). Unfractionated blood products are optionally obtained directly from a donor or retrieved from cryopreservative storage.

Sources for HSC expansion also include AGM, ESC and iPSC. ESC are well-known in the art, and may be obtained from commercial or academic sources (Thomson et al., 282 Sci. 1145-47 (1998)). iPSC are a type of pluripotent stem cell artificially derived from a non-pluripotent cell, typically an adult somatic cell, by inducing a “forced” expression of certain genes (Baker, Nature Rep. Stem Cells (Dec. 6, 2007); Vogel & Holden, 23 Sci. 1224-25 (2007)). ESC, AGM, and iPSC according to the present invention may be derived from animal or human sources. As discussed herein, the AGM stem cell is a cell that is born inside the aorta, and colonies the fetal liver. Signaling pathways can increase AGM stem cells make it likely that these pathways will increase HSC in ESC.

As discussed above, administration of the NO signaling modulator affects the HSC population. Enhanced NO signaling may occur in an HSC itself and/or in an HSC supporting cell. As used herein, the term HSC supporting cell refers to cells naturally found in the vicinity of one or more HSCs such that factors released by HSC supporting cells reach the HSC by diffusion, for example. HSC supporting cells include, but are not limited to, lymphoreticular stromal cells. Lymphoreticular stromal cells as used herein include, but are not limited to, all cell types present in a lymphoid tissue which are not lymphocytes or lymphocyte precursors or progenitors. Thus, lymphoreticular stromal cells include, osteoblasts, epithelial cells, endothelial cells, mesothelial cells, dendritic cells, splenocytes, and macrophages. Lymphoreticular stromal cells also include cells that would not ordinarily function as lymphoreticular stromal cells, such as fibroblasts, which have been genetically altered to secrete or express on their cell surface the factors necessary for the maintenance, growth or differentiation of hematopoietic stem cells, including their progeny. Lymphoreticular stromal cells are optionally derived from the disaggregation of a piece of lymphoid tissue. Such cells are capable of supporting in vitro the maintenance, growth or differentiation of hematopoietic stem cells, including their progeny. By lymphoid tissue it is meant to include bone marrow, peripheral blood (including mobilized peripheral blood), umbilical cord blood, placental blood, fetal liver, embryonic cells (including embryonic stem cells), AGM derived cells, and lymphoid soft tissue. Lymphoid soft tissue as used herein includes, but is not limited to, tissues such as thymus, spleen, liver, lymph node, skin, tonsil, adenoids and Peyer's patch, and combinations thereof.

Lymphoreticular stromal cells provide the supporting microenvironment in the intact lymphoid tissue for the maintenance, growth or differentiation of hematopoietic stem cells, including their progeny. The microenvironment includes soluble and cell surface factors expressed by the various cell types which comprise the lymphoreticular stroma. Generally, the support which the lymphoreticular stromal cells provide is characterized as both contact-dependent and non-contact-dependent.

Lymphoreticular stromal cells, for example, are autologous (self) or non- autologous (non-self, e.g., heterologous, allogeneic, syngeneic or xenogeneic) with respect to hematopoietic stem cells. Autologous, as used herein, refers to cells from the same subject. Allogeneic, as used herein, refers to cells of the same species that differ genetically. Syngeneic, as used herein, refers to cells of a different subject that are genetically identical to the cell in comparison. Xenogeneic, as used herein, refers to cells of a different species. Lymphoreticular stroma cells are obtained, for example, from the lymphoid tissue of a human or a non-human subject at any time after the organ/tissue has developed to a stage (i.e., the maturation stage) at which it can support the maintenance, growth or differentiation of hematopoietic stem cells. The lymphoid tissue from which lymphoreticular stromal cells are derived usually determines the lineage-commitment hematopoietic stem cells undertake, resulting in the lineage-specificity of the differentiated progeny.

The co-culture of hematopoietic stem cells (and progeny thereof) with lymphoreticular stromal cells, usually occurs under conditions known in the art (e.g., temperature, CO₂ and O₂ content, nutritive media, duration, etc.). The time sufficient to increase the number of cells is a time that can be easily determined by a person skilled in the art, and varies depending upon the original number of cells seeded. The amounts of hematopoietic stem cells and lymphoreticular stromal cells initially introduced (and subsequently seeded) varies according to the needs of the experiment. The ideal amounts are easily determined by a person skilled in the art in accordance with needs.

As used throughout, by a subject is meant an individual. Thus, subjects include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject is optionally a mammal such as a primate or a human.

The subject referred to herein is, for example, a bone marrow donor or an individual with or at risk for depleted or limited blood cell levels. Optionally, the subject is a bone marrow donor prior to bone marrow harvesting or a bone marrow donor after bone marrow harvesting. The subject is optionally a recipient of a bone marrow transplant. The methods described herein are particularly useful in subjects that have limited bone marrow reserve such as elderly subjects or subjects previously exposed to an immune depleting treatment such as chemotherapy. The subject, optionally, has a decreased blood cell level or is at risk for developing a decreased blood cell level as compared to a control blood cell level. As used herein the term control blood cell level refers to an average level of blood cells in a subject prior to or in the substantial absence of an event that changes blood cell levels in the subject. An event that changes blood cell levels in a subject includes, for example, anemia, trauma, chemotherapy, bone marrow transplant and radiation therapy. For example, the subject has anemia or blood loss due to, for example, trauma. The subject optionally has depleted bone marrow related to, for example, congenital, genetic or acquired syndrome characterized by bone marrow loss or depleted bone marrow. Thus, the subject is optionally a subject in need of hematopoeisis. Optionally, the subject is a bone marrow donor or is a subject with or at risk for depleted bone marrow.

HSC manipulation is useful as a supplemental treatment to chemotherapy or radiation therapy. For example, HSC are localized into the peripheral blood and then isolated from a subject that will undergo chemotherapy, and after the therapy the cells are returned. Thus, the subject is a subject undergoing or expected to undergo an immune cell-depleting treatment such as chemotherapy, radiation therapy or serving as a donor for a bone marrow transplant. Bone marrow is one of the most prolific tissues in the body and is therefore often the organ that is initially damaged by chemotherapy drugs and radiation. The result is that blood cell production is rapidly destroyed during chemotherapy or radiation treatment, and chemotherapy or radiation must be terminated to allow the hematopoietic system to replenish the blood cell supplies before a patient is re-treated with chemotherapy. Therefore, as described herein, HSCs or blood cells made by the methods described herein are optionally administered to such subjects in need of additional blood cells.

Provided are pharmaceutical compositions comprising one or more NO signaling modulators or combinations thereof and a least one pharmaceutically acceptable excipient or carrier. By pharmaceutically acceptable is meant a material that is not biologically or otherwise undesirable, i.e., the material may be administered to a subject or cell, without causing undesirable biological effects or interacting in a deleterious manner with the other components of the pharmaceutical composition in which it is contained. The carrier or excipient is selected to minimize degradation of the active ingredient and to minimize adverse side effects in the subject or cell.

The compositions are formulated in any conventional manner for use in the methods described herein. Administration is via any route known to be effective by one of ordinary skill For example, the compositions is administered orally, parenterally (e.g., intravenously), by intramuscular injection, by intraperitoneal injection, transdermally, extracorporeally, intranasally or topically.

For oral administration, the compositions take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets are coated by methods well known in the art. Liquid preparations for oral administration take the form of, for example, solutions, syrups or suspensions, or they may be presented as a dry product for constitution with water or other suitable vehicle before use. Such liquid preparations are prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations optionally contain buffer salts, flavoring, coloring and sweetening agents as appropriate.

The compositions are formulated for parenteral administration by injection, e.g., by bolus injection or continuous infusion. Formulations for injection are presented in unit dosage form, e.g., in ampules or in multi-dose containers, with or without an added preservative. The compositions take such forms as suspensions, solutions or emulsions in oily or aqueous vehicles, and may contain formulatory agents such as suspending, stabilizing and/or dispersing agents. Alternatively, the active ingredient is in powder form for constitution with a suitable vehicle, e.g., sterile pyrogen-free water, before use. In general, water, a suitable oil, saline, aqueous dextrose (glucose polymer), and related sugar solutions and glycols such as propylene glycol or polyethylene glycols are suitable carriers for parenteral solutions. Solutions for parenteral administration contain, for example, a water soluble salt of the active ingredient, suitable stabilizing agents and, if necessary, buffer substances. Antioxidizing agents such as sodium bisulfate, sodium sulfite or ascorbic acid, either alone or combined, are suitable stabilizing agents. Also citric acid and its salts and sodium ethylenediaminetetraacetic acid (EDTA) are optionally used. In addition, parenteral solutions optionally contain preservatives such as benzalkonium chloride, methyl- or propyl-paraben and chlorobutanol. Suitable pharmaceutical carriers are described in REMINGTON: SCI. & PRACTICE PHARMACY (21st Ed., Troy, ed., Lippicott Williams & Wilkins 2005).

The compositions are optionally formulated as a depot preparation. Such long acting formulations are optionally administered by implantation. Thus, for example, the compositions are formulated with suitable polymeric or hydrophobic materials (for example as an emulsion in an acceptable oil) or ion exchange resins, or as sparingly soluble derivatives, for example, as a sparingly soluble salt. The compositions are applied to or embedded with implants concurrent with or after surgical implant.

Additionally, standard pharmaceutical methods are employed to control the duration of action. These include control release preparations and appropriate macromolecules, for example, polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation are adjusted in order to control release. Optionally, the agent is incorporated into particles of polymeric materials such as polyesters, polyamino acids, hydrogels, poly (lactic acid) or ethylenevinylacetate copolymers. In addition to being incorporated, these agents are optionally used to trap the compound in microcapsules.

A composition for use in the methods described herein is optionally formulated as a sustained and/or timed release formulation. Such sustained and/or timed release formulations are made by sustained release means or delivery devices that are well known to those of ordinary skill in the art. The compositions are used to provide slow or sustained release of one or more of the active ingredients using, for example, hydropropylmethyl cellulose, other polymer matrices, gels, permeable membranes, osmotic systems, multilayer coatings, microparticles, liposomes, microspheres or a combination thereof to provide the desired release profile in varying proportions. Suitable sustained release formulations are selected for use with the compositions described herein. Thus, single unit dosage forms suitable for oral administration, such as, but not limited to, tablets, capsules, gelcaps, caplets, powders, that are adapted for sustained release are used.

The compositions are optionally delivered by a controlled-release system. For example, the composition is administered using intravenous infusion, an implantable osmotic pump, liposomes, or other modes of administration. A controlled release system is placed in proximity to the target. For example, a micropump delivers controlled doses directly into bone, thereby requiring only a fraction of the systemic dose (see, e.g., Goodson, 2 MEDICAL APPL. CONTROLLED RELEASE, 115-138 (1984)). In another example, a pharmaceutical composition is formulated with a hydrogel (see, e.g., U.S. Pat. No. 5,702,717; No. 6,117,949; No. 6,201,072).

Optionally, it is desirable to administer the composition locally, i.e., to the area in need of treatment. For example, the composition is administered by injection into the bone marrow of a long bone, for example. Local administration is achieved, for example, by local infusion during surgery, topical application (e.g., in conjunction with a wound dressing after surgery), injection, catheter, suppository, or implant. An implant is of a porous, non-porous, or gelatinous material, including membranes, such as sialastic membranes, or fibers.

The pharmaceutical compositions described herein are administered by any conventional means available for use in conjunction with pharmaceuticals, either as individual therapeutic active ingredients or in a combination of therapeutic active ingredients. They are optionally administered alone, but are generally administered with a pharmaceutical carrier selected on the basis of the chosen route of administration and standard pharmaceutical practice.

The HSC modulators described herein are provided in a pharmaceutically acceptable form including pharmaceutically acceptable salts and derivatives thereof. The term pharmaceutically acceptable form refers to compositions including the compounds described herein that are generally safe, relatively non-toxic and neither biologically nor otherwise undesirable. These compositions optionally include pharmaceutically acceptable carriers or stabilizers that are nontoxic to the cell or subject being exposed thereto at the dosages and concentrations employed. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (Uniqema, United Kingdom), polyethylene glycol (PEG), and PLURONICS™ (BASF, Germany).

The term pharmaceutically acceptable acid salts and derivatives refers to salts and derivatives of the prostaglandins and prostaglandin receptor agonists described herein that retain the biological effectiveness and properties of the prostaglandins and prostaglandin receptor agonists as described, and that are not biologically or otherwise undesirable. Pharmaceutically acceptable salts are formed, for example, with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The chemical stability of a composition comprising a HSC modulator or a pharmaceutically acceptable salt or ester thereof is enhanced by methods known to those of skill in the art. For example, an alkanoic acid ester of a polyethoxylated sorbitol (a polysorbate) is added to a composition containing a prostaglandin in an amount effective to enhance the chemical stability of the HSC modulator.

The dosage administered is a therapeutically effective amount of the compound sufficient to result in promoting an increase in HSC numbers varies depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired.

Toxicity and therapeutic efficacy of such compounds is determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it is expressed as the ratio LD₅₀/ED₅₀.

The data obtained from the cell culture assays and animal studies are optionally used in formulating a range of dosage for use in humans. The dosage of such compounds lies preferably within a range of circulating concentrations that include the ED₅₀ with little or no toxicity. The dosage varies within this range depending upon the dosage form employed and the route of administration utilized. For any compound used in the provided methods, the therapeutically effective dose is estimated initially from cell culture assays. A dose is formulated in animal models to achieve a circulating plasma concentration range that includes the IC₅₀ (i.e., the concentration of the test compound which achieves a half-maximal inhibition of symptoms) as determined in cell culture. Such information is used to more accurately determine useful doses in humans or other subjects. Levels in plasma are measured, for example, by high performance liquid chromatography.

The dosage ranges for the administration of the compositions are those large enough to produce the desired effect in which the symptoms of the disorder are affected. The dosage are not so large as to cause adverse side effects, such as unwanted cross-reactions and anaphylactic reactions. Dosage varies and is administered in one or more dose administrations daily for one or several days. For example, the pharmaceutical compositions comprising one or more prostaglandins and/or prostaglandin receptor agonists can be administered by systemic injection once or twice a day for one or several days. The compositions are administered daily as necessary for weeks, months or even years as necessary. Optionally the compositions are administered weekly or monthly. Thus, the compositions are administered once or more times daily for at least about eight days, at least about ten days, at least about twelve days, at least about fourteen days, at least about twenty days, at least about thirty days or more or any number of days in between.

Also provided herein is a pack or kit comprising one or more containers filled with one or more of the ingredients (e.g., a NO signaling agonist) described herein. Thus, for example, a kit described herein comprises one or more NO signaling agonists (e.g., SNAP). Such kits optionally comprise solutions and buffers as needed or desired. The kit optionally includes an expanded population of HSC made by the methods described, or can contain containers or compositions for making an expanded population of HSC. Optionally associated with such pack(s) or kit(s) are instructions for use.

Also provided is a kit for providing an effective amount of a HSC modulator to increase or decrease HSCs in a subject ion need thereof comprising one or more doses of the NO agonist for use over a period of time, wherein the total number of doses of the NO agonist in the kit equals the effective amount of the NO agonist or combination thereof sufficient to increase HSCs in a subject. The period of time is from about one to several days or weeks or months. Thus, the period of time is from at least about five, six, seven, eight, ten, twelve, fourteen, twenty, twenty-one or thirty days or more or any number of days between one and thirty. The doses of HSC modulator are administered once, twice, three times or more daily or weekly. The kit provides one or multiple doses for a treatment regimen

A kit for providing an effective amount of a NO signaling pathways agonist for expanding a population of HSCs is described. The kit comprises one or more aliquots NO signaling agonists or combinations thereof for administration to HSC or a mixture of HSC and HSC-supporting cells over a period of time, wherein the aliquots equal the effective amount of the NO signaling agents required to expand the population of HSC. The period of time is from about one to several hours or one to several days. The amount of NO signaling agonist (e.g., SNAP) or combination thereof is administered once, twice, three times or more daily or weekly and the kit provides one or multiple aliquots.

Optionally, the methods and kits comprise effective amounts of HSC modulator(s) for administering to the subject the HSC modulator(s) thereof in a second or subsequent regime for a specific period of time. The second or subsequent period of time, like the first period of time, is, for example, at least one or more days, weeks or months, such as, for example, at least four, five, six, seven, eight, nine, ten, eleven, twelve, fourteen, twenty one, or thirty days or any number of days between. In the methods herein, the interval between the first treating period and the next treating period is optionally, for example, days, weeks, months or years. Thus, the interval between the first period of time and the next period of time is, for example, at least four, five, six, seven, eight, nine, ten, eleven, twelve, fourteen, twenty one, or twenty eight days or in number of days between. This treating schedule is repeated several times or many times as necessary. Such schedules are designed to correlated with repeated bone marrow depleting events such as repeated chemotherapy treatments or radiation therapy treatments. Optionally, a drug delivery device or component thereof for administration is included in a kit. Disclosed are materials or steps in a method, compositions, and components that are used for, are used in conjunction with, are used in preparation for, or are products of the disclosed method and compositions. These and other materials are disclosed herein, and it is understood that when combinations, subsets, interactions, groups, etc. of these materials or steps are disclosed that, while specific reference of each various individual and collective combinations and permutation of these materials or steps may not be explicitly disclosed, each is specifically contemplated and described herein.

For example, if NO signaling agonist, such as SNAP, is disclosed and discussed and a number of modifications that can be made to a number of molecules are discussed, each and every combination and permutation of SNAP and the modifications that are possible are specifically contemplated unless specifically indicated to the contrary. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed. Optional or optionally means that the subsequently described event or circumstance may or may not occur, and that the description includes instances where said event or circumstance occurs and instances where it does not.

Definitive hematopoietic stem cells (HSCs) that are capable of self-renewal and production of all mature blood lineages arise during embryogenesis. Both the timing of HSC induction and the gene programs regulating this process are well conserved across vertebrate species (Orkin & Zon, 132 Cell 631-44 (2008)). Additionally, factors that affect HSC specification during embryogenesis often similarly function in HSC maintenance and/or recovery after marrow injury. The identification of factors that regulate HSC induction during embryogenesis is of significant interest.

Murine transplantation studies revealed that adult-type longterm repopulating (LTR) HSCs arise in the AGM region between embryonic day 10.5 (e10.5) and e11.5 (Dzierzak & Speck, 9 Nat. Immunol. 129-36 (2008)). Transplantable HSCs localize to the ventral wall of the dorsal aorta and express phenotypic markers of mesenchymal, endothelial, or hematopoietic cell types. Runx1, commonly affected in childhood and adult leukemia (Downing et al., 81 Blood 2860-65 (1993); Golub et al., 92 P.N.A.S. USA 4917-21 (1995)), is required for the formation of functional HSCs (North et al., 126 Devel. 2563-75 (1999), North et al., 16 Immun. 661-72 (2002); Wang et al., 93 P.N.A.S. USA3444-49 (1996)); its expression is highly conserved across vertebrate species (Orkin & Zon, 2008). Based on the functional conservation of aorta-gonadsmesonephros (AGM) hematopoiesis from fish to man, an evolutionary advantage or necessity for the production of stem cells within the aorta must exist. To identify genes that regulate HSC formation, we conducted a chemical genetic screen for regulators of runx1/cmyb+ cells in the zebrafish AGM at 36 hours postfertilization (hpf). Previously, PGE2 was identified as a potent regulator of both HSC induction and marrow repopulation across vertebrate species (North et al., 447 Nature 1007-11 (2007)). The wnt pathway similarly regulates stem cell production during embryogenesis, and genetically interacts with PGE2 (Goessling et al., 136 Cell 1136-47 (2008a)). The identification of novel regulators of this process will aid in connecting the complex network of signaling pathways that control both HSC development during embryogenesis and marrow regulation in the adult.

Nitric oxide (NO) plays a key role in the regulation of vascular tone, angiogenesis, and endothelial migration (Davies, 75 Physiol. Rev. 519-60 (1995); Lucitti et al., 134 Devel. 3317-26 (2007)). As HSCs are derived from hemogenic endothelial cells within the dorsal aorta, NO produced locally in endothelial cells could link blood flow and HSC formation. NO has been detected in blood cells and extends replating ability in hematopoietic culture, presumably by maintaining HSCs in a quiescent state in vitro (Krasnov et al., 14 Mol. Med. 141-49 (2008)). Although NO function in the adult hematopoietic stroma is thought to have a positive effect on hematopoiesis, data from knockout mice imply that NO production is detrimental to hematopoietic repopulation and recovery after injury (Michurina et al., 10 Mol. Ther. 241-48 (2004)); this effect, however, may be due to NO-related superoxide complexes induced by irradiation (Epperly et al., 35 Exp. Hematol. 137-45 (2007)). The role of NO in HSC induction in the vertebrate embryo is currently uncharacterized.

The embodiments of the present invention provide for a diverse group of compounds that regulate blood flow affect the production of runx1/cmyb+ HSCs. In general, compounds that increased blood flow enhanced HSC number, whereas chemicals that decreased blood flow diminished HSCs. silent heart (sih) embryos that lack a heartbeat and fail to establish blood circulation had impaired HSC formation. Compounds that increase NO production could modify HSC formation when exposure occurred prior to the initiation of circulation and rescue HSC production in sih mutants. Inhibition of NO production blocked the inductive effect of several blood flow modulators on HSCs, suggesting that NO serves as the connection between blood flow and HSC formation. In the mouse, NO synthase 3 (Nos3; eNos) is expressed in AGM endothelium and hematopoietic clusters, and marks LTR-HSCs. Intrauterine Nos inhibition reduced transplantable HSCs; similar results were found for the Nos3^{-/-} knockout mice. The present invention provides a direct link between the initiation of circulation and the onset of hematopoiesis within the AGM, and identifies NO signaling as a conserved regulator of HSC development.

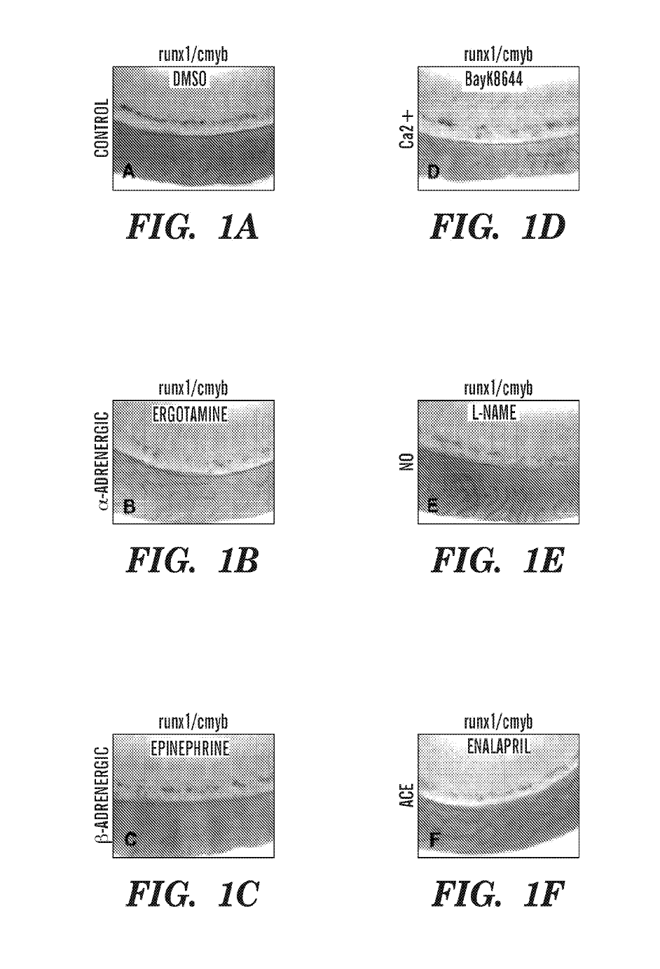

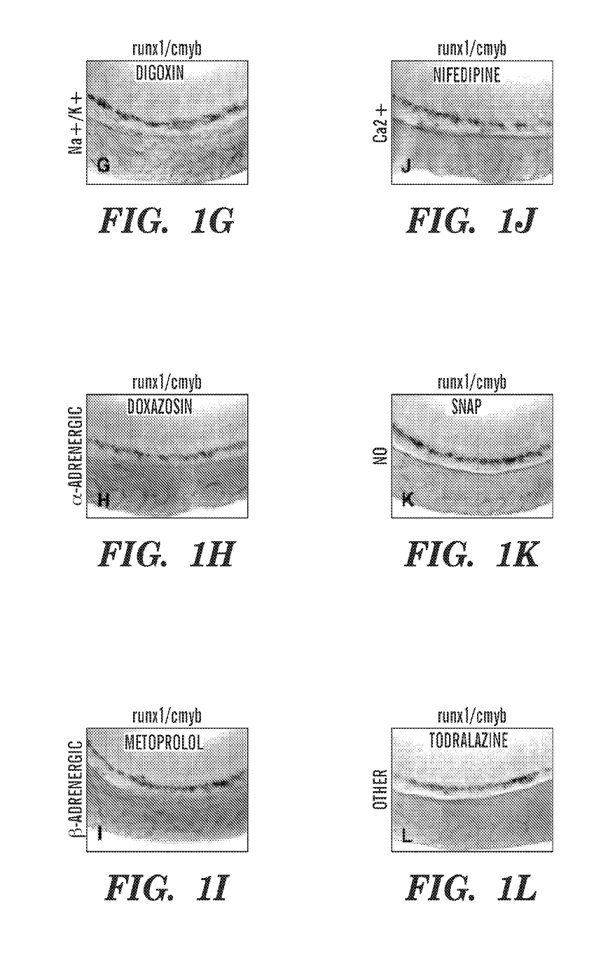

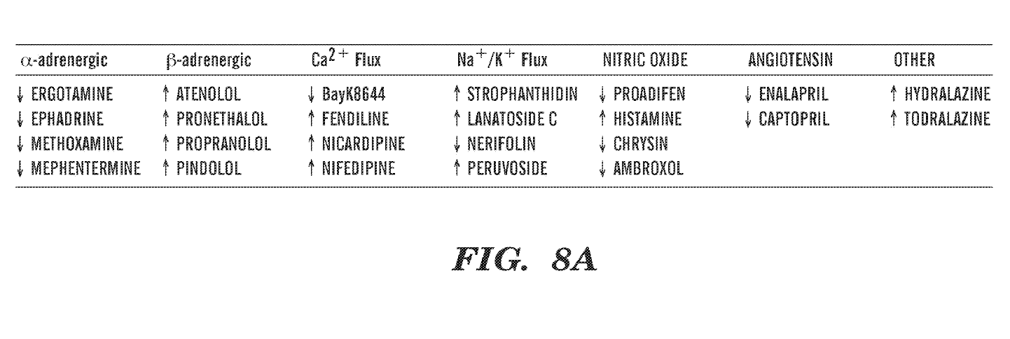

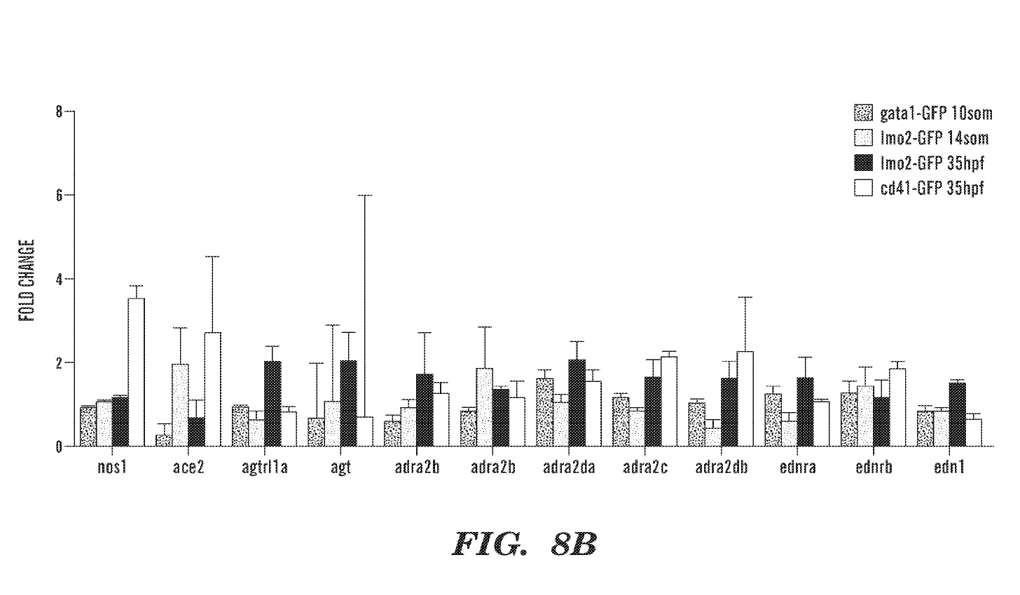

More specifically, modulators of blood flow that regulate HSC formation were identified using a chemical genetic screen that identified regulators of AGM HSC formation (North et al., 2007). Of the chemicals found to regulate runx1 and cmyb coexpression by in situ hybridization at 36 hpf, several were known modulators of heartbeat and blood flow. These compounds were categorized into distinct classes on the basis of their hemodynamic mechanism of action (FIG. 8). Well-established agonists and antagonists of each category were secondarily screened for effects on HSCs (FIGS. 1A-1L). The adrenergic signaling pathways affect both cardiac and vascular physiology. Exposure to the α1-adrenergic blocker doxasozin (10 μM) enhanced HSCs (58 increased [inc]/86 scored), while the α-agonist ergotamine (10 μM) decreased HSC number (FIGS. 1B and 1H, 42 decreased [dec]/82). Similarly, the β1-adrenergic blocker metoprolol increased (49 inc/77) and the β1-agonist epinephrine decreased runx1/cmyb staining (FIGS. 1C and 1I, 40 dec/70).

Changes in electrolyte balance potently regulate cardiac and vascular reactivity. The Ca²⁺-channel blocker nifedipine enhanced HSC formation (48 inc/85), while BayK8644 diminished HSC number (FIGS. 1D and 1J, 34 dec/79). The cardiac glycoside digoxin, a modulator of Na⁺/K⁺ fluxes, also increased HSCs (FIG. 1G, 56 inc/79).

NO is a well-established direct regulator of vascular tone and reactivity, thereby influencing blood flow. The NO donor S-nitroso-N-acetyl-penicillamine (SNAP) (10 μM) caused a significant increase in HSC development (69 inc/93). In contrast, the Nos inhibitor N-nitro-L-arginine methyl ester (L-NAME) (10 μM) diminished runx1/cmyb expression (FIGS. 1E and 1K, 58 dec/90). Exposure to the angiotensin-converting enzyme (ACE) inhibitor enalapril decreased HSC number (FIG. 1F, 42 dec/81). These findings were corroborated by qPCR for runx1 (FIG. 1M).

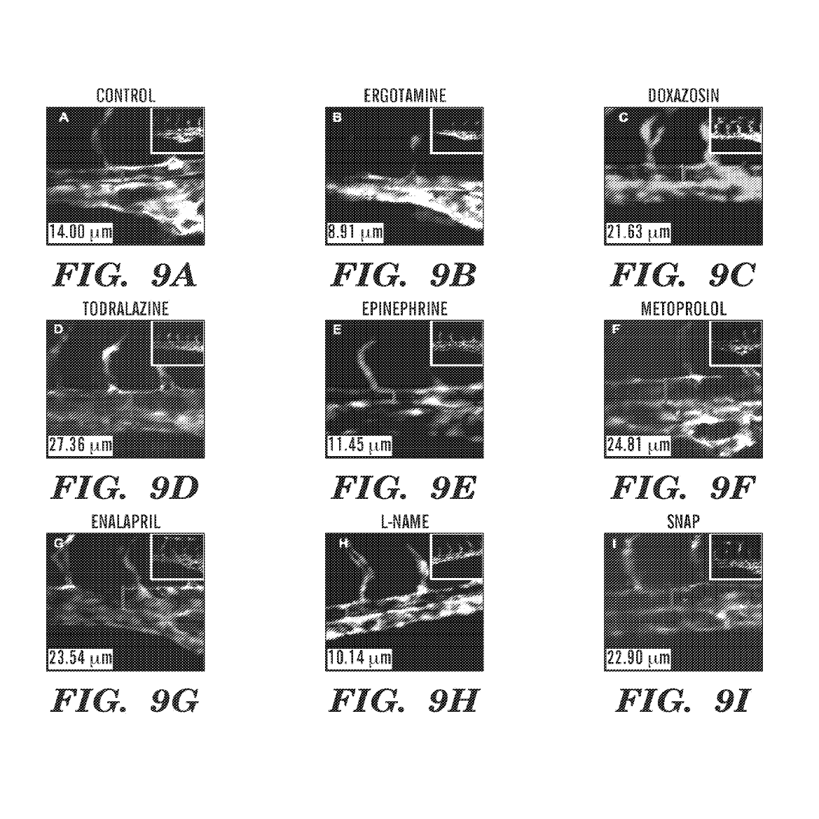

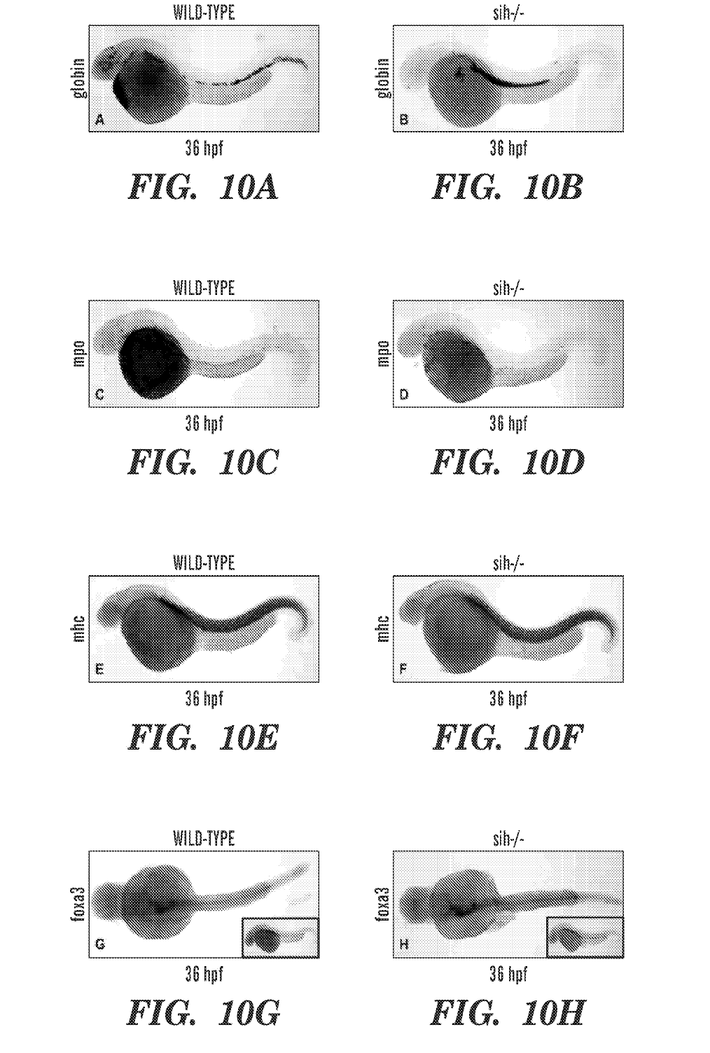

Conserved vascular responses of each chemical class were demonstrated by in vivo confocal microscopy of fli:GFP; gata1:dsRed transgenic zebrafish (n=5/compound) at 36 hpf (FIGS. 1N and 9) (Eddy, 142 Comp. Biochem. Physiol. A Mol. Integr. Physiol. 221-30 (2005)). These data correlated with prior zebrafish studies (Fritsche et al., 279 μm. J. Physiol. Regul. Integr. Comp. Physiol. R2200-07 (2000)). Vasodilation of the artery and vein was accompanied by increased passage of total blood volume, as seen by digital motion analysis of gata1⁺ red blood cells (RBCs); vasoconstriction caused RBCs to traverse only in single file. Together with the in situ hybridization studies, these experiments reveal that increases in vessel diameter typically were coincident with increased runx1 expression, and vice versa.

Microarray analysis of sorted cell populations isolated during various stages of embryogenesis has been used to document cell-type and developmental specificity of genes of interest (North et al., 2007; Weber et al., 106 Blood 521-30 (2005)). Components of the NO (nos1), angiotensin (ace2, agtrl1a, agt), and adrenergic signaling (adra2b, adra2 da, adra2c) pathways are expressed in the HSC compartment (FIG. 8B). Most were more highly expressed during the definitive wave of hematopoiesis after the onset of the heartbeat and circulation, consistent with their role in regulating hemodynamic homeostasis. These data confirm that vascular tone and flow-modifying components are present and responsive to chemical manipulation in the AGM and imply that modulation of blood flow could have a significant impact on HSC formation during embryonic development.

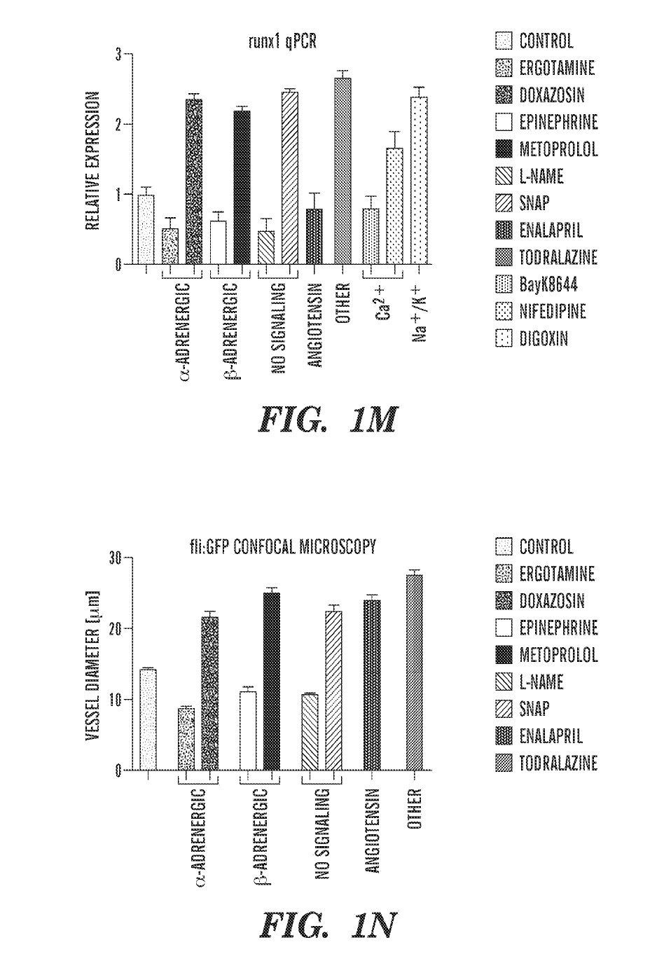

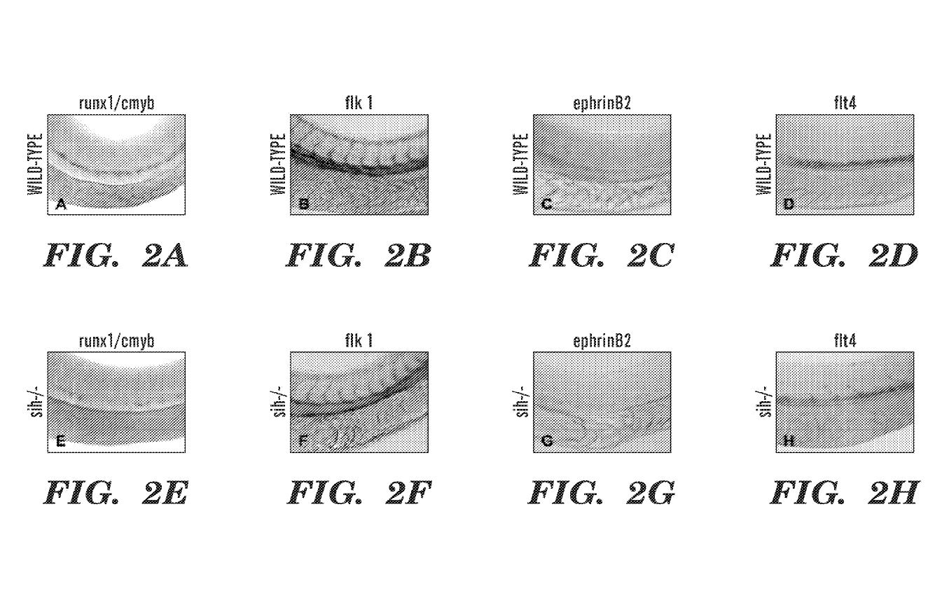

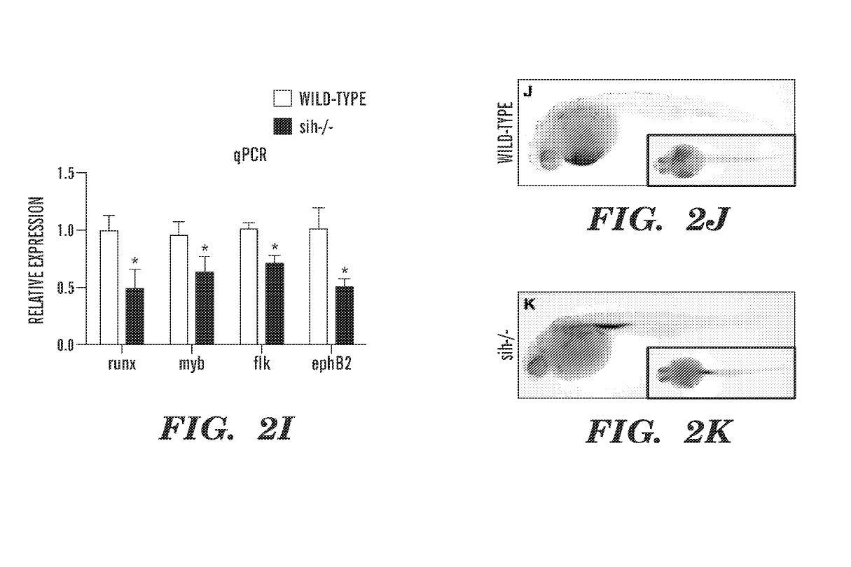

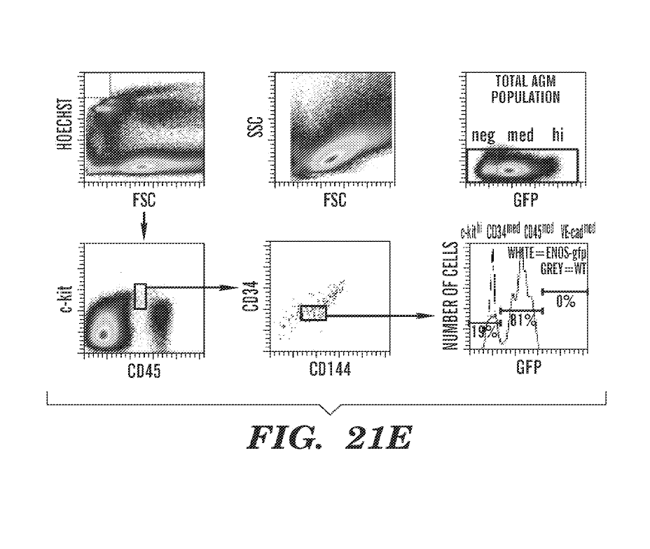

Absence of a heartbeat causes failures in definitive HSC development. In Zebrafish, the occurrence of vigorous blood circulation through the tail is coincident with HSC formation in this region. In order to establish the importance of blood flow for initiation of HSC formation, we examined sih mutant Zebrafish embryos, which lack a heartbeat due to a mutation in cardiac troponin T (Sehnert et al., 31 Nat. Genet. 106-10 (2002)) (FIGS. 2J and 2K). In fact, runx1/cmyb expression was dramatically reduced in sih^{-/-} embryos (FIGS. 2A and 2E, 69 dec/77). In contrast, the vascular marker flk1 was minimally affected (FIGS. 2B and 2F), consistent with previous observations (Isogai et al., 130 Devel. 5281-90 (2003)). A marker of arterial identity, ephrinB2, was reduced in sih embryos (FIGS. 2C and 2G, 55 dec/74), while expression of the venous marker flt4 was increased (FIGS. 2D and 2H, 33 inc/61). These results were confirmed by qPCR (FIG. 21, p<0.05, n=3). In contrast, the erythroid marker globin and the myeloid marker myeloperoxidase (mpo) show distribution differences due to lack of blood circulation in sih mutants, but no gross quantitative changes (FIGS. S3A-S3H). Myosin heavy chain (mhc), a marker of somitogenesis, and the endodermal progenitor marker foxa3 were also not affected. These data demonstrate that the absence of a beating heart and subsequent failure to establish circulation specifically impairs arterial identity and HSC formation.

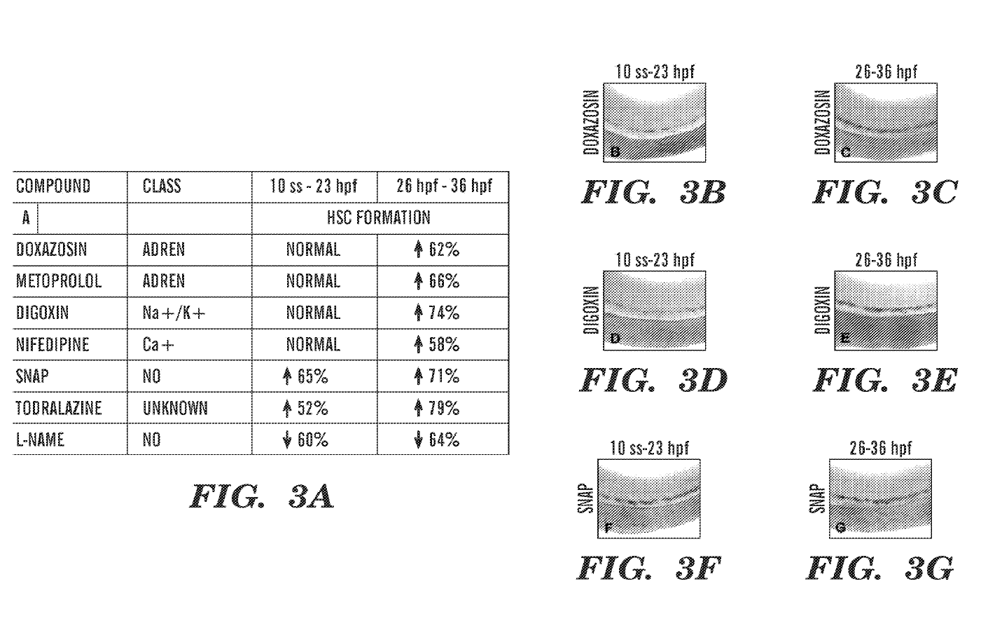

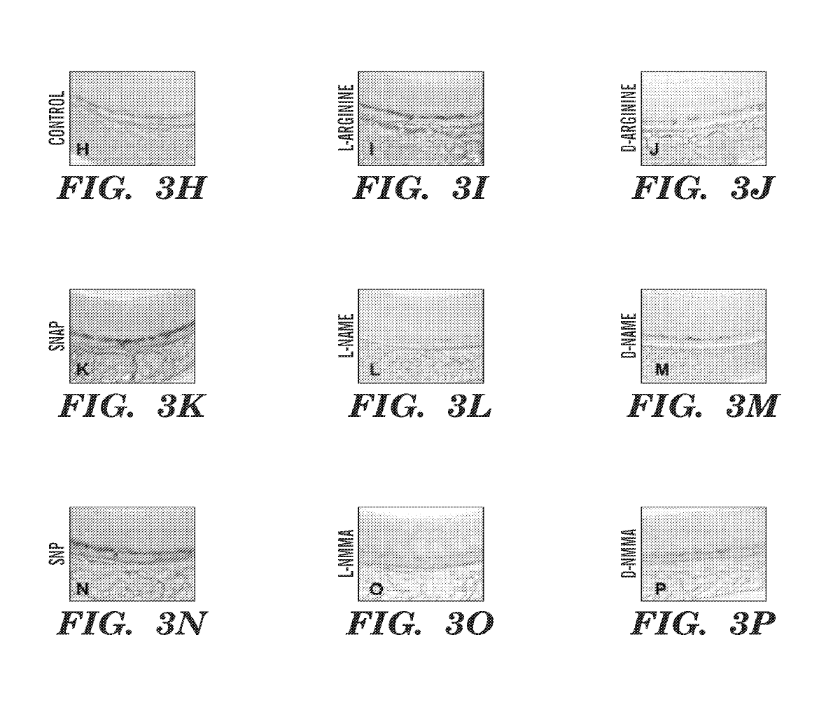

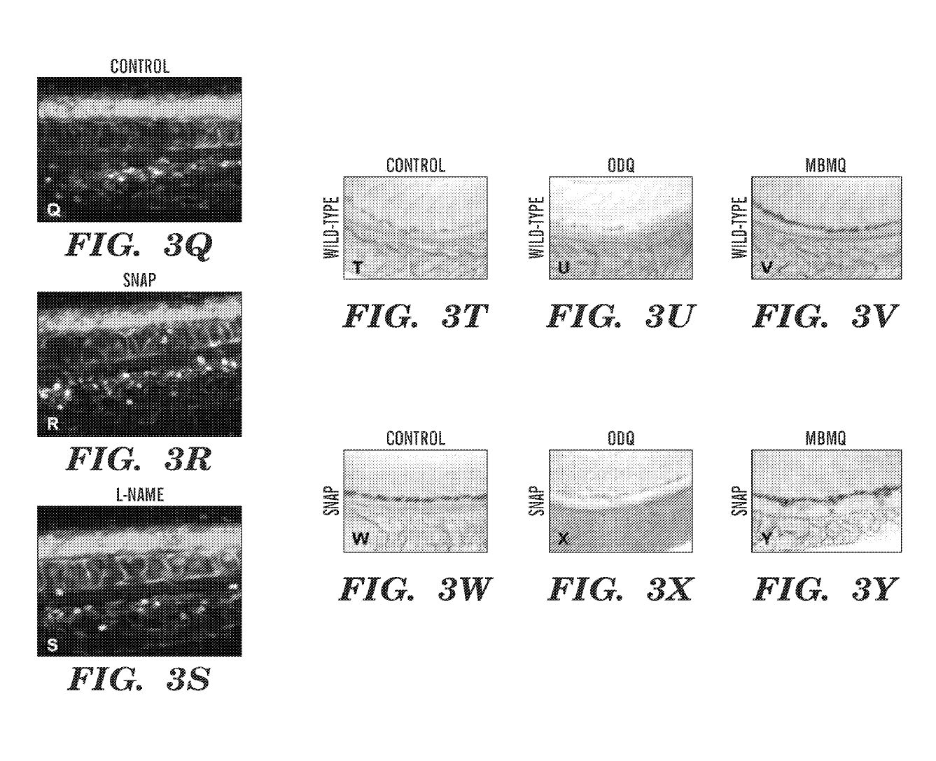

NO signaling can affect HSC formation prior to the initiation of blood flow. To further investigate the role of circulation in the initiation of HSC development, Zebrafish embryos were exposed to blood flow-modulating agents either before (10 somites-23 hpf) or after (26-36 hpf) the onset of heartbeat and assessed HSC development at 36 hpf. All compounds examined increased HSC formation when used after the heartbeat began (FIGS. 3A, 3C, 3E, and 3G).

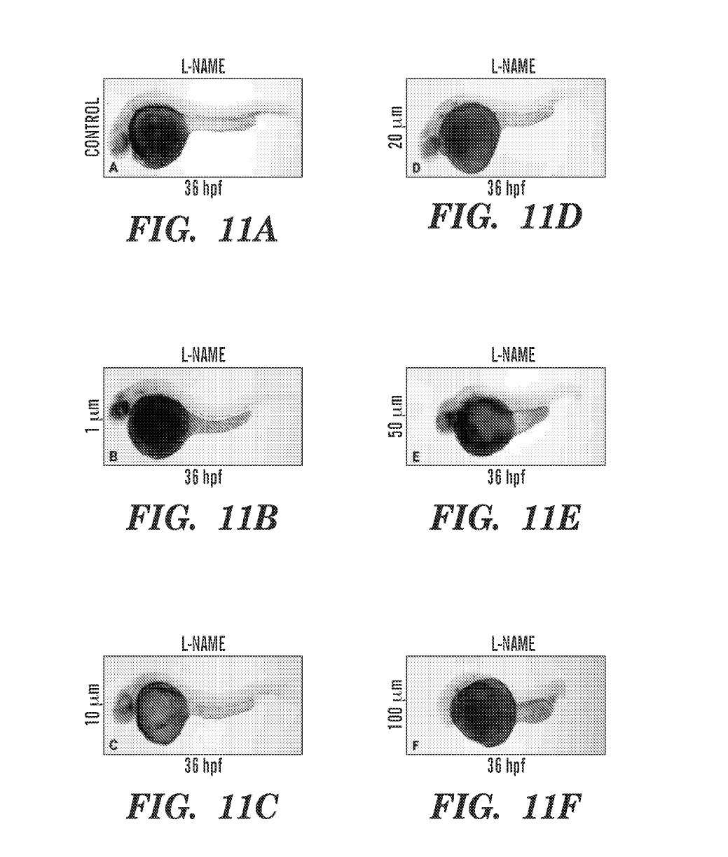

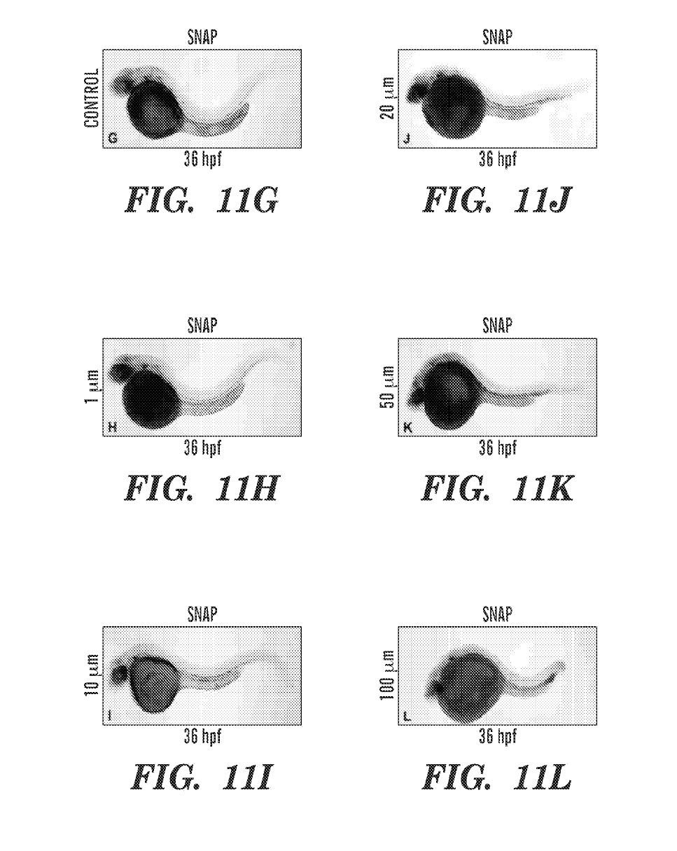

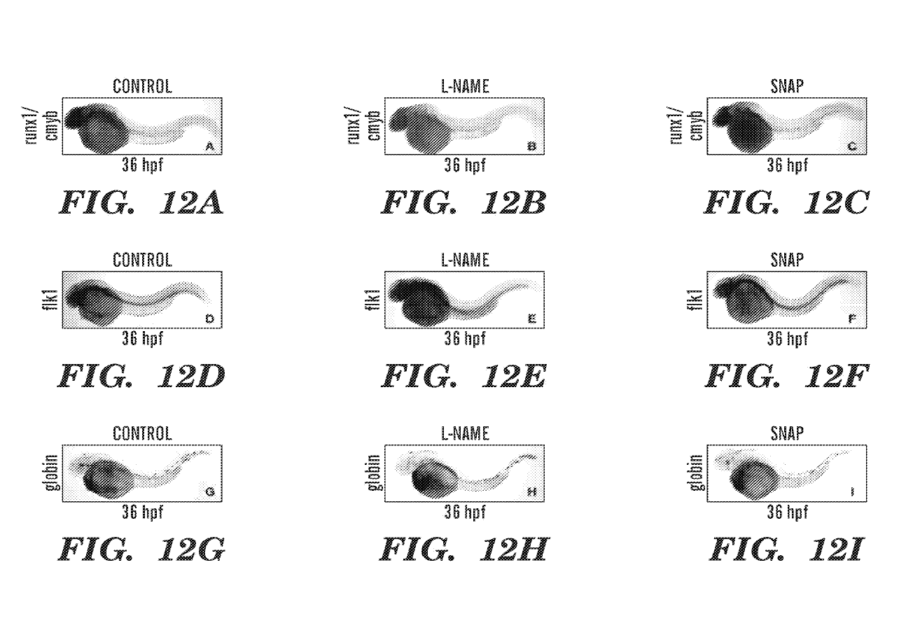

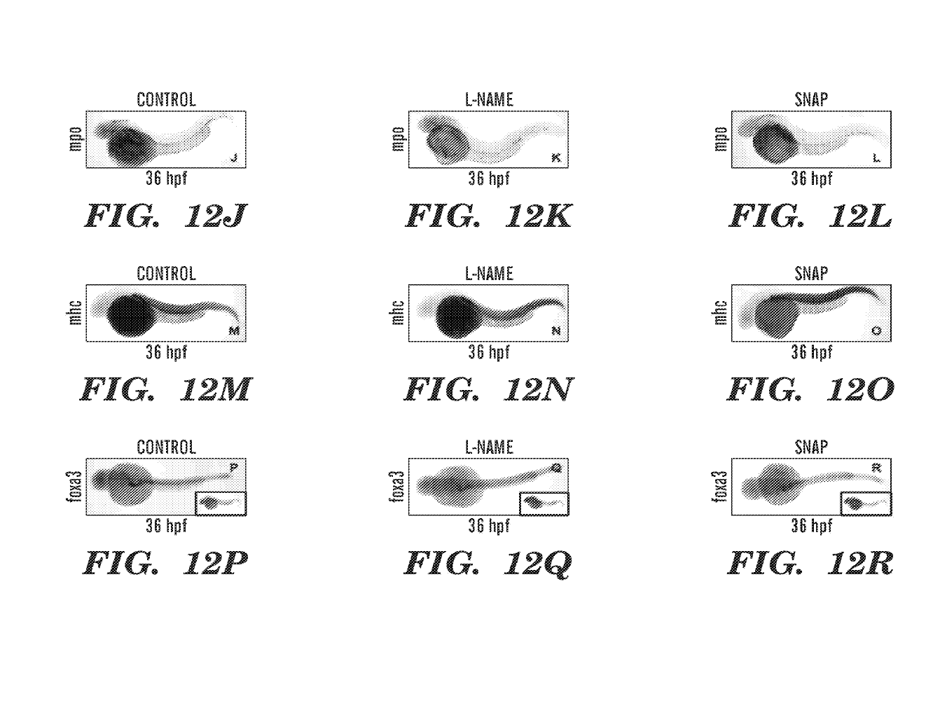

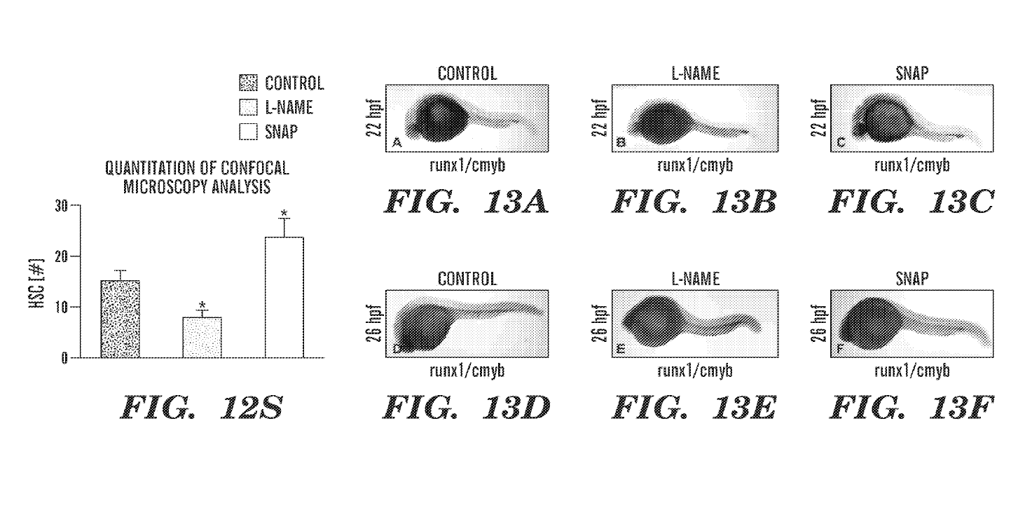

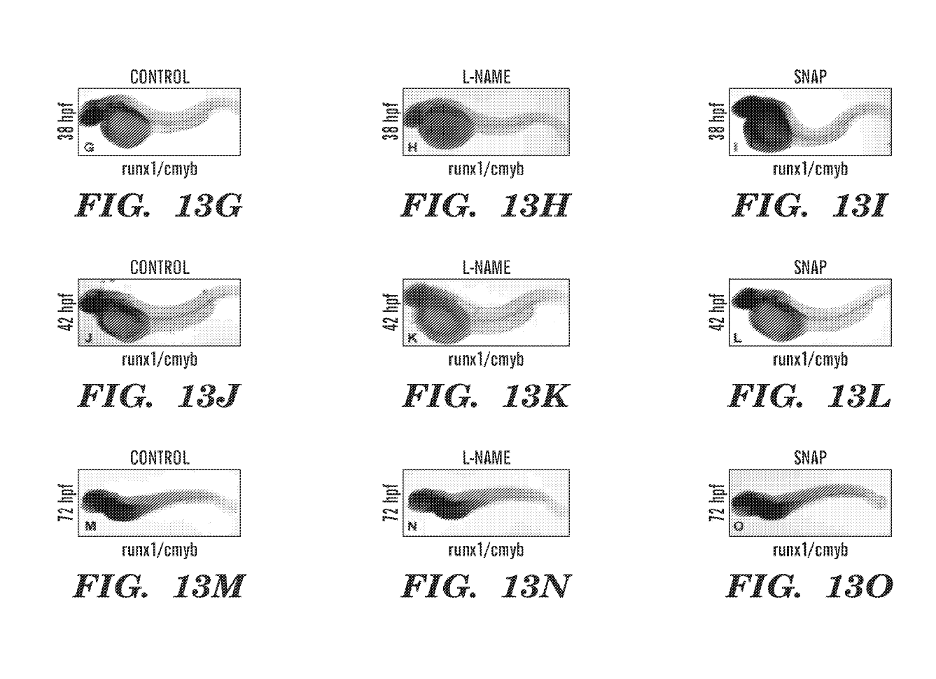

In contrast, only SNAP was capable of enhancing HSC number at 36 hpf when treatment was completed before the heartbeat was established (FIGS. 3B, 3D, and 3F). Conversely, the NO inhibitor L-NAME reduced HSC formation when treatment occurred prior to the initiation of the heartbeat (FIG. 3A). The effects of L-NAME and SNAP were dose dependent over a range of 1-100 μM (FIGS. 11A-11L) and specific to the HSC compartment, with mild effects on the vasculature, but not on globin, mpo, mhc, or foxa3 expression (FIGS. 12A-12R). Additionally, changes were only observed during the definitive hematopoietic wave and were maintained into larval stages (FIGS. 13A-130). In addition to SNAP (FIG. 3K, 18 inc/25), NO donors sodium nitroprusside (SNP; FIG. 3N, 20 inc/31) and L-arginine (L-arg; FIG. 3I, 15 inc/25) (Pelster et al., 2005; Pyriochou et al., 2006) enhanced runx1/cmyb expression (FIGS. 3I, 3K, and 3N), while the nonspecific nos inhibitor, N-monomethyl-L-arg acetate (L-NMMA; FIG. 3O, 17 dec/29) diminished HSCs like L-NAME (FIG. 3L, 16 dec/26). Inactive D-enantiomers had no effect (FIGS. 3J, 3M, and 3P).

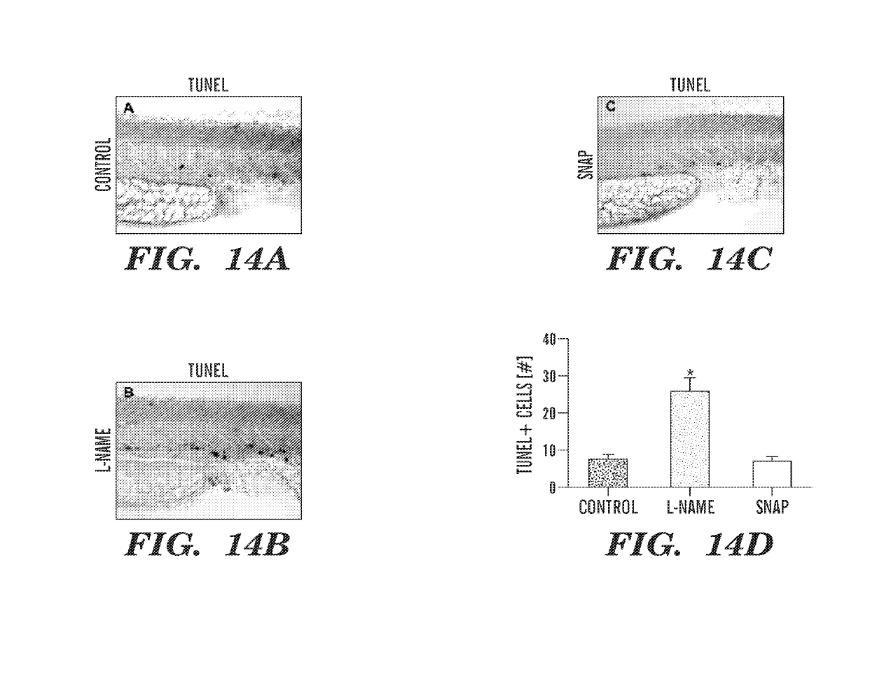

Whether modification of runx1/cmyb expression correlated with a quantifiable effect on HSC number was assessed using cmyb:GFP; lmo2:dsRed reporter fish (North et al., 2007). Confocal microscopy revealed increased HSC numbers after SNAP exposure, and a reduction after L-NAME treatment (FIGS. 3Q-3S and 12S, p<0.001, n=5). TUNEL analysis indicated that L-NAME could affect HSC by induction of apoptosis (FIGS. 14A-14D). These analyses indicate that HSC modulation by the majority of flow-modifying compounds requires the establishment of blood circulation and that NO signaling is the mediator of blood flow in this process.

To clarify that the effect of flow on HSCs was mediated by NO signaling, downstream components of the NO signaling cascade were manipulated chemically. The soluble guanyl cyclase inhibitor 1H-oxadiazolo-quinoxalin-1-one (ODQ) prevents cGMP formation in response to NO signaling; it regulates vascular remodeling and blood flow in zebrafish in a dose- and time-dependent manner (Pyriochou et al., 2006). ODQ (10 mM) caused a profound decrease in HSCs (FIGS. 3T and 3U, 27 dec/43) and also blocked the effects of SNAP (FIGS. 3W and 3X, 8 inc/38). Phosphodiesterase V (PDEV) converts cGMP to GTP. The PDEV inhibitor 4-{[3′,4′-methylene-dioxybenzyl]amino}-6-methoxyquinazoline (MBMQ, 10 μM) increased HSCs (FIG. 3V, 35 inc/43) and further enhanced the effects of SNAP (FIG. 3Y, 40 inc/46). These data highlight the specificity of cGMP as a downstream effector of NO signaling in HSC formation.

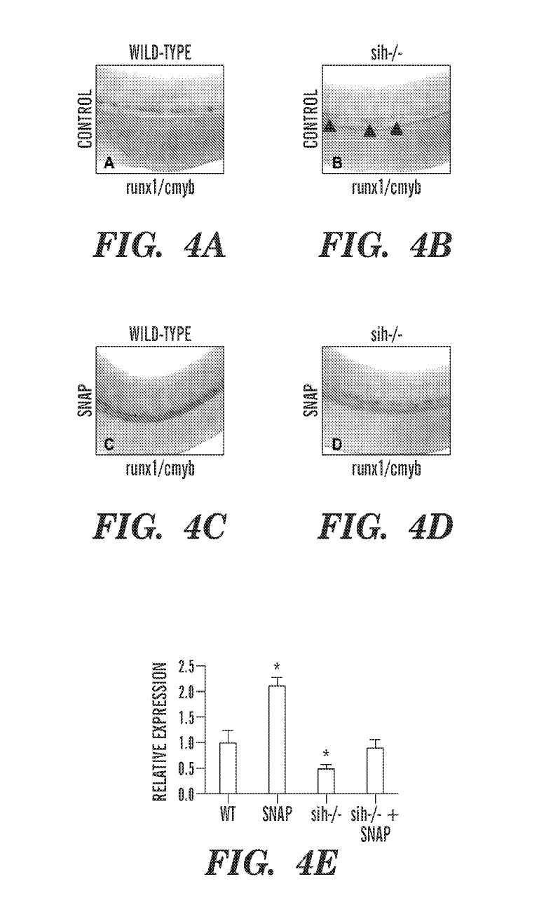

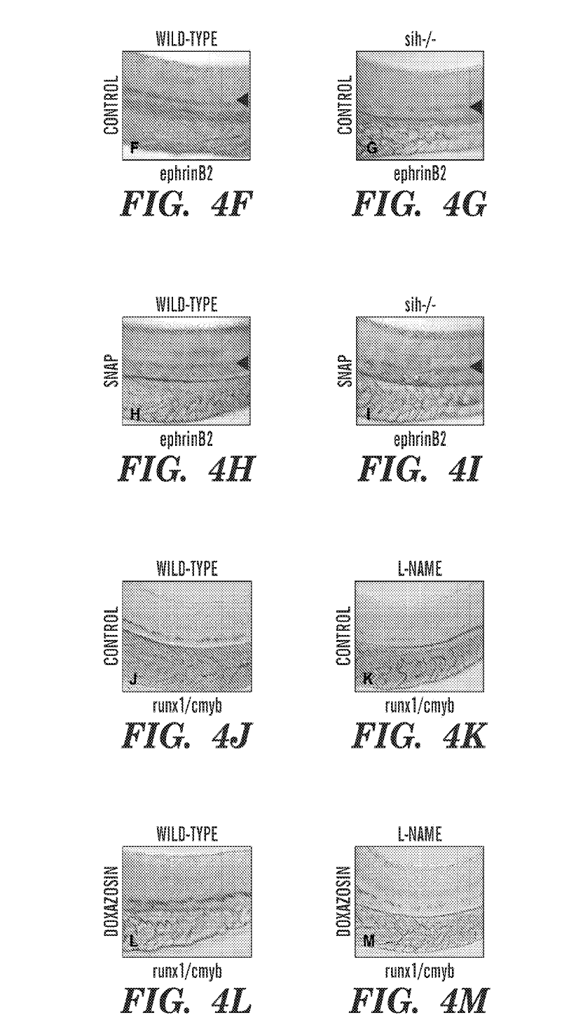

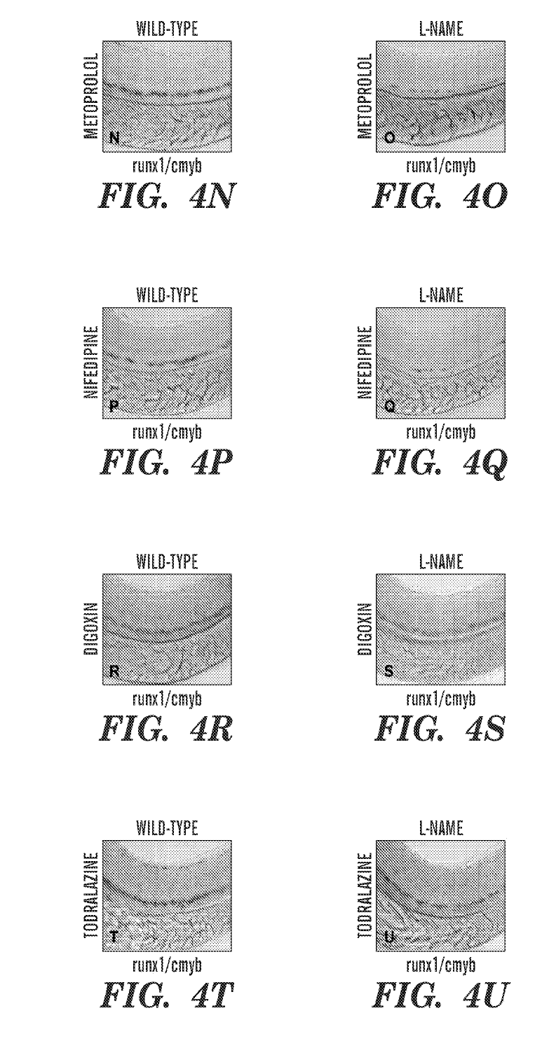

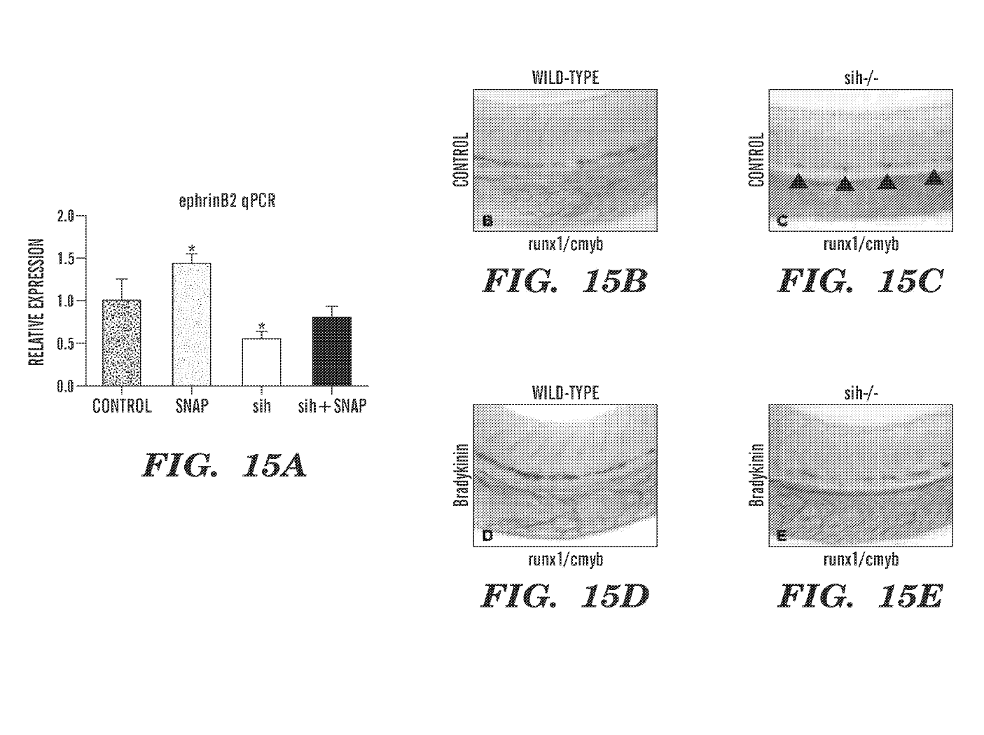

To confirm a direct role for NO in HSC induction, sih embryos were exposed to SNAP. SNAP rescued runx1/cmyb expression toward wild-type (WT) levels in the majority of sih embryos examined (FIGS. 4A-4D, 31 normal/51). These results were confirmed by qPCR (FIG. 4E). SNAP also normalized ephB2 defects in sih mutants (FIGS. 4F-4I, 18 inc/27; FIG. 15A). L-arg and SNP, as well as bradykinin, a potent vasodilator that stimulates NO production, increased HSCs, and rescued the sih hematopoietic defect (FIGS. 15B-15E).

To further characterize the relationship between blood flow, NO signaling, and HSC induction, WT embryos were exposed concomitantly to flow-modifying drugs and L-NAME (10 μM). Because the majority of flow-regulating compounds that enhance HSCs also cause vasodilation and increase total blood flow through the aorta, they may directly trigger NO production by alterations in sheer stress, pulsatile flow, or soluble signaling components. L-NAME treatment prevented the increase in HSC formation caused by most compounds tested (FIGS. 4J-4U). These data further point to NO signaling as the direct link between blood flow and HSC development.

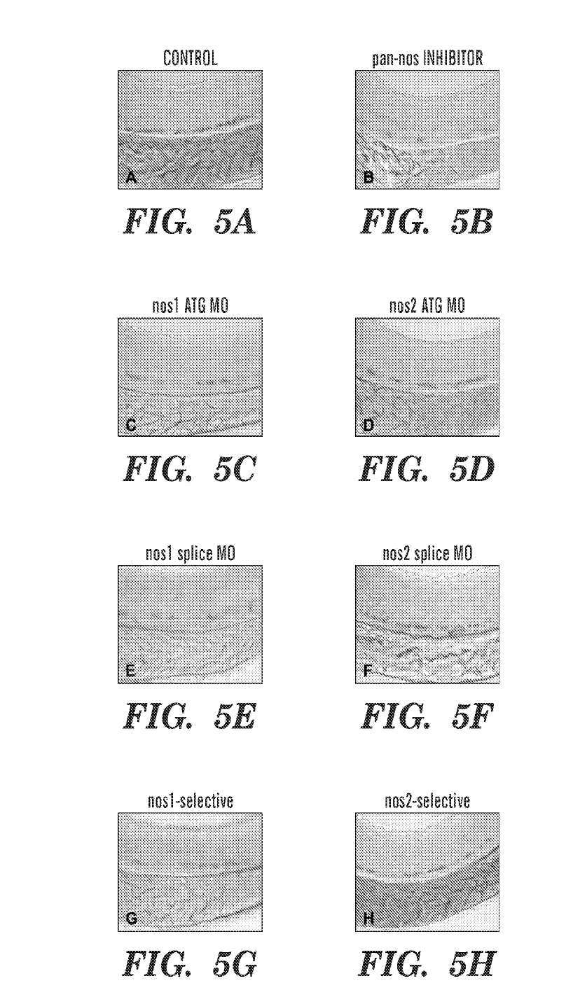

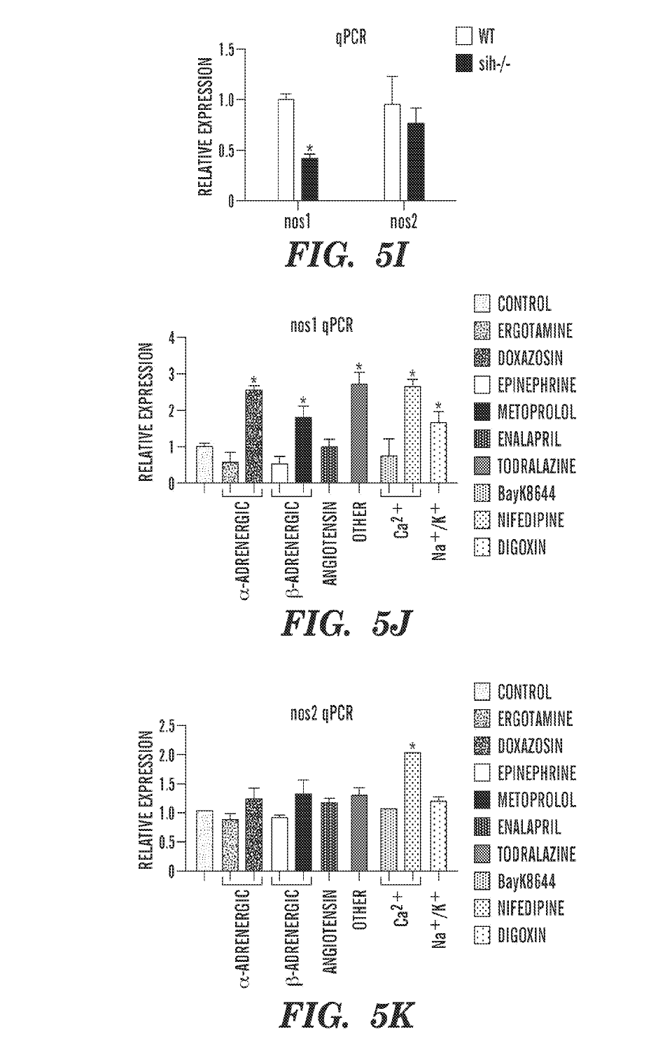

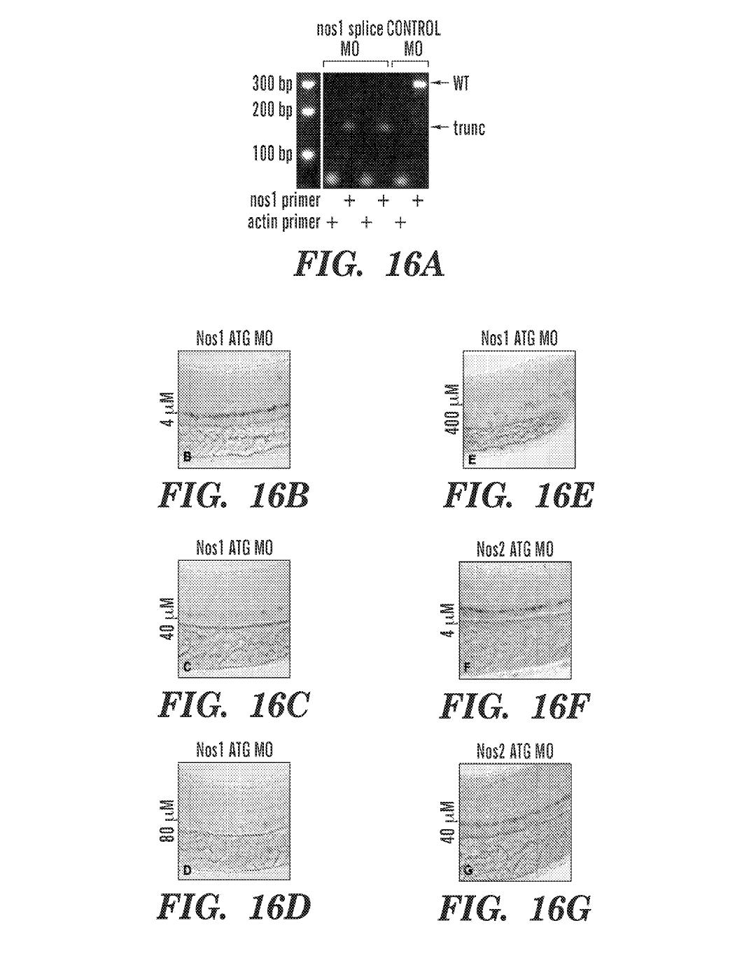

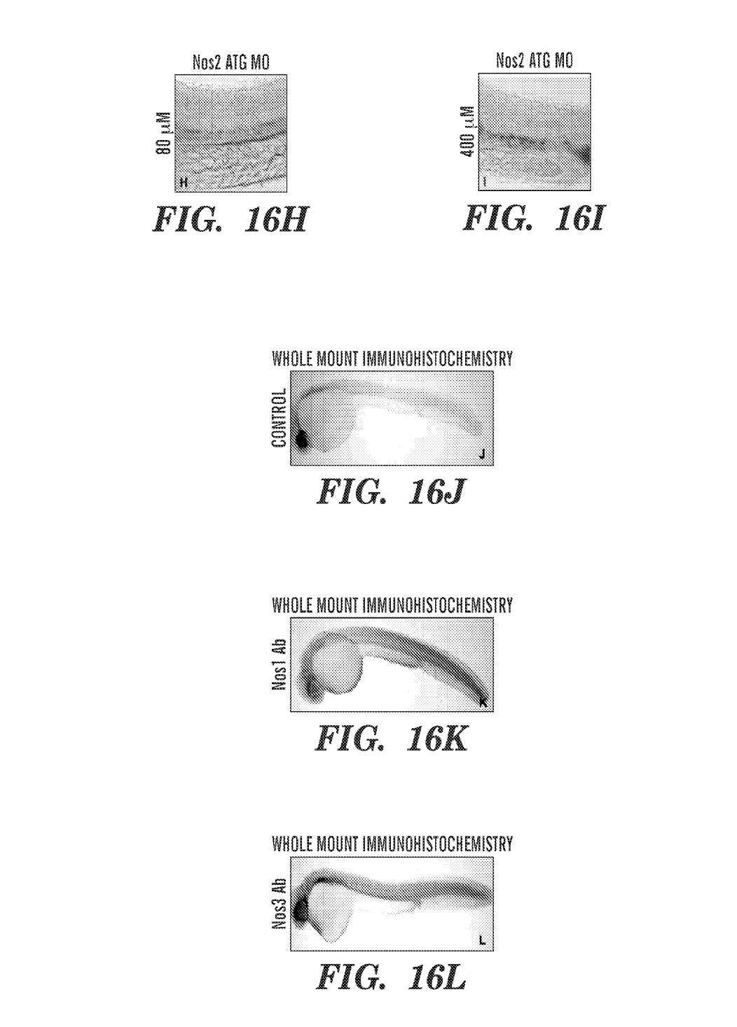

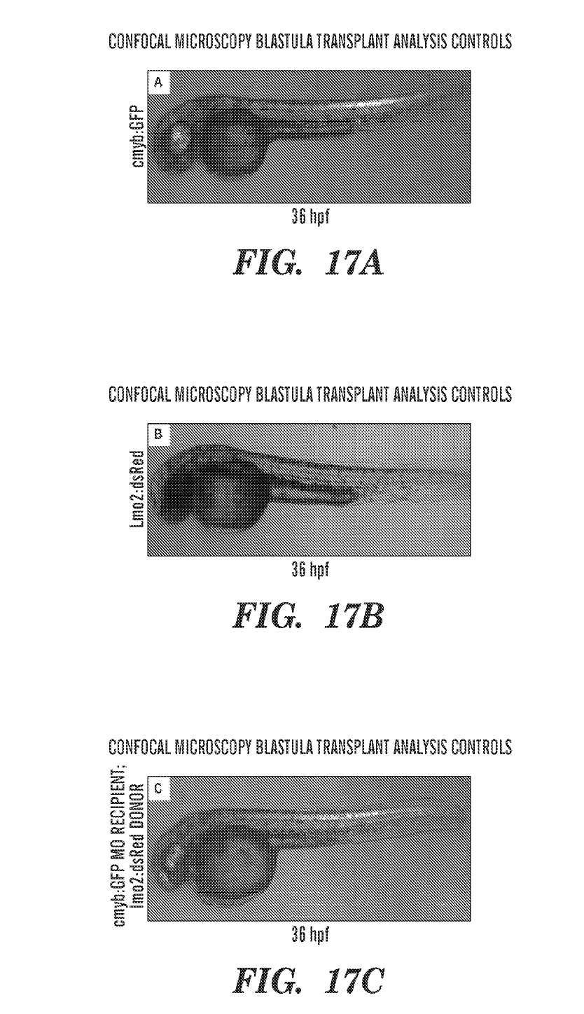

Zebrafish lack genomic evidence for endothelial NO synthase (enos, nos3), but there is eNos immunoreactivity in the tail region, where HSCs develop (FIGS. 16J-16L) (see also Pelster et al., 142 Comp. Biochem. Physiol. A Mol. Integr. Physiol. 215-20 (2005)). Phylogenetic and genomic examination demonstrate that neuronal nos (nnos, nos1) (Poon et al., 3 Gene Expr. Patterns 463-66 (2003)), and nos3 (enos) are highly related. Morpholino antisense oligonucleotide (MO) knockdown of nos1 had a profound dose-dependent impact on HSC development (FIGS. 5A, 5C, and 5E; 63 dec/89 ATG MO, 48 dec/64 splice MO; FIGS. 17A-17E), whereas knockdown of nos2 (inducible nos, inos) did not affect runx1/cmyb expression (FIGS. 5D, and 5F; 9 dec/98 ATG MO, 10 dec/65; FIGS. 16F-16I). The potent effect of nos1 was confirmed by chemical inhibition of NO synthesis (FIGS. 5B, 5G, and 5H): selective inhibition of nos1 by S-methyl-L-thiocitrulline (10 μM; 30 dec/44) severely diminished HSC number, whereas the nos2 (inos)-specific inhibitor 1400W (10 μM; 4 dec/49) only minimally affected HSCs. These data suggest that nos1 (nnoslenos) is required for HSC formation in Zebrafish, which is supported by nos1 expression in both endothelial cells and HSCs (FIG. 8B). Interestingly, in sih^{-/-} embryos, nos1 was significantly decreased (FIG. 5I, p<0.001); in contrast, nos2 was not significantly changed. Further, nos1, but not nos2, was significantly altered in response to chemical alteration of blood flow (FIGS. 5J and 5K, p<0.003). These data indicate that nos1 is the functionally relevant connection between blood flow and HSC development.

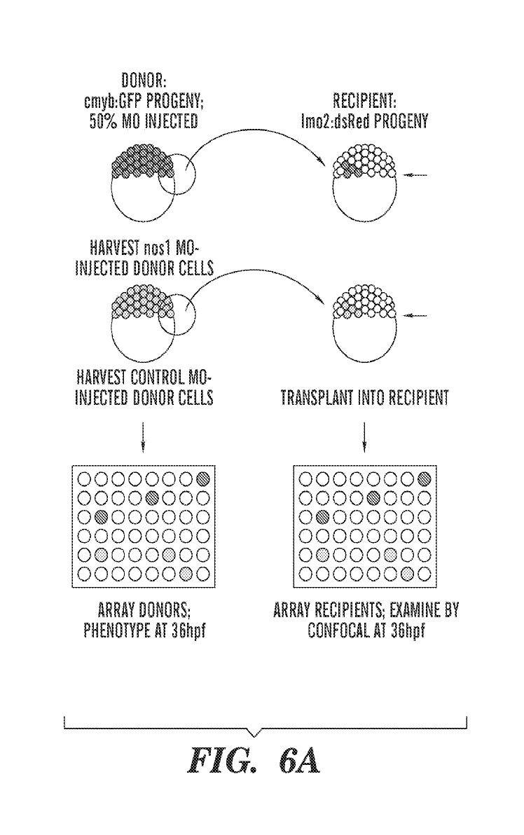

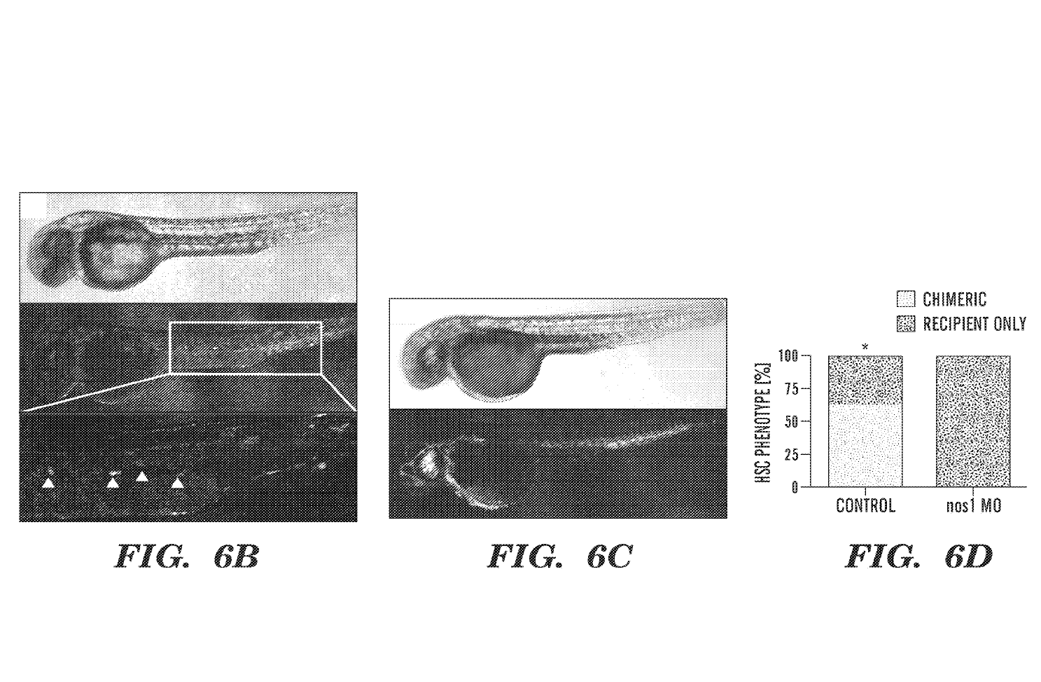

Cell autonomy and delineation of the role of NO signaling in the HSC and surrounding hematopoietic niche was explored using a blastula transplant strategy. Cells harvested from cmyb:GFP embryos injected with control or nos1 MO were transplanted at the blastula stage into lmo2:dsRed recipients. In this transplant scheme, donor-derived HSCs appeared green (FIG. 17A) in the red fluorescent endothelial/HSC compartment (FIGS. 6A and 17B). Of the embryos examined, 62.5% had GFP+HSC formation derived from control-injected donor cells (FIGS. 6B and 6D), whereas none of the nos1 MO injected donor cells gave rise to green HSCs (FIGS. 3C and 3D, p=0.0065); successful transplants were indicated by the contribution of cmyb+donor cells to the recipient eye. In a reciprocal experiment, uninjected lmo2:dsRed donor cells contributed to endothelial and HSC development in cmyb:GFP recipients (FIG. 17C), particularly after MO knockdown in the recipient. These experiments demonstrate that nos1 acts in a cell-autonomous manner in the hemogenic endothelial cell.

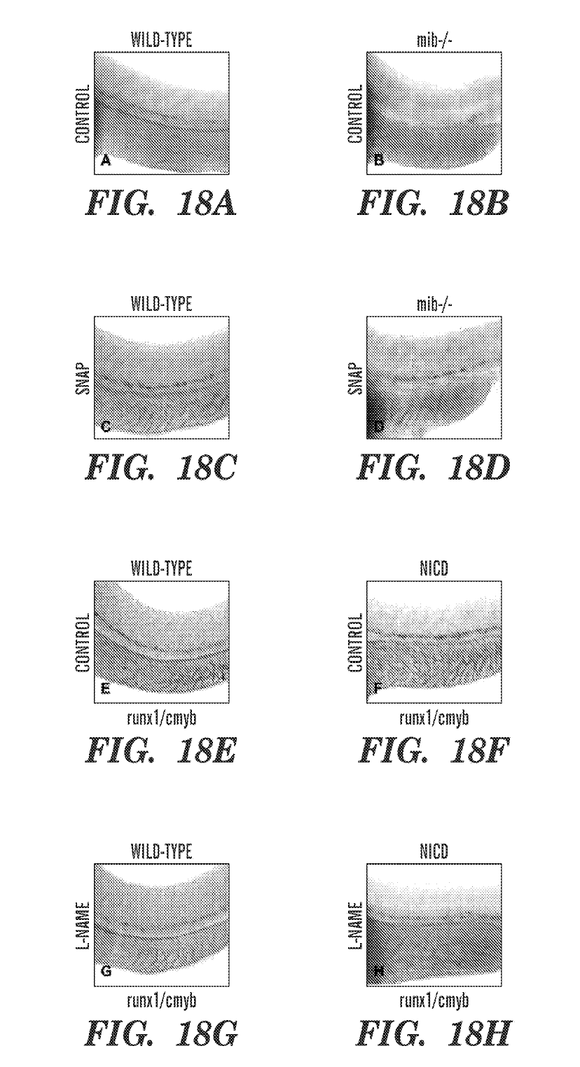

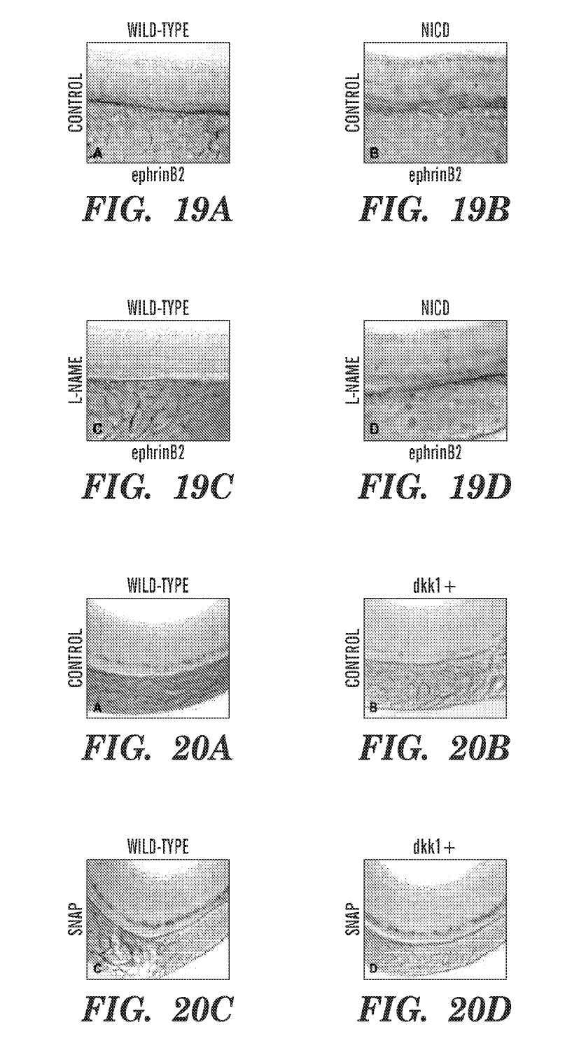

Developmental signaling pathways interact with NO in HSC formation. More specifically, developmental regulators such as the notch and wnt pathways have been linked to HSC formation and selfrenewal (Burns et al., 19 Genes Devel. 2231-42 (2005); Goessling et al., 2008a; WO 2007/112084). Due to the effect of NO on HSC specification and expansion, potential interaction with notch and wnt signaling was examined. The notch pathway influences arterial/venous identity and functions upstream of runx1 in HSC specification; mindbomb (mib) mutants lack HSCs because of a deficiency of notch signaling (Burns et al., 2005) (33 dec/47). SNAP rescued HSC formation in these mutants (FIGS. 18A-18D; 27 normal/43). Transgenic zebrafish embryos expressing an activated form of the notch intracellular domain (NICD) exhibit enhanced HSC numbers (55 inc/62); L-NAME blocked the HSC increase (FIGS. 18E-18H; 16 inc/63) and inhibited NICD-mediated elevation of ephB2 expression in the aorta (FIGS. 19A-19D). These studies imply that NO functions downstream of notch in regulation of arterial identity and/or in HSC induction.

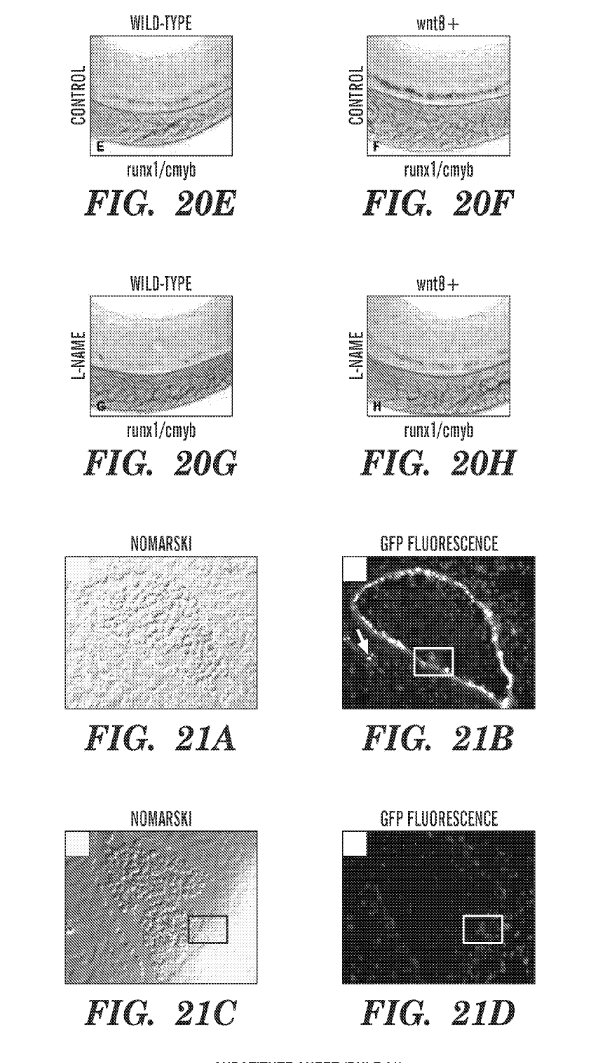

Recent studies have shown that modulation of the wnt pathway affects HSCs (Goes sling et al., 2008a; Reya et al., 423 Nature 409-14 (2003)). Heat-shock-inducible transgenic zebrafish embryos expressing negative (dkk) and positive (wnt8) regulators of wnt signaling were used to evaluate the interaction between the wnt and NO signaling cascades (Goes sling et al., 320 Devel. Biol. 161-74 (2008b)). Induction of dkk reduced HSC number (16 dec/8), and was rescued by SNAP (FIGS. 20A-20D; 5 dec/33). In contrast, wnt8 enhanced HSC formation (22 inc/30), which was blocked by L-NAME (FIGS. 20E-20H; 9 inc/28). In support of these findings, previous studies have shown an interaction of both the notch and wnt pathways with NO, although the directionality of these interactions varied (Du et al., 66 Cancer Res. 7024-31 (2006); Ishimura et al., 128 Gastroenterology 1354-68 (2005); Prevotat et al., 131 Gastroenterology 1142-52 (2006)).

The relationship between NO and HSC induction is also present in mammals. To document a role for NO in murine HSC formation, the Nos3:GFP expression in the AGM was examined. Histological sections of e11.5 embryos showed endothelial cells lining the dorsal aorta expressing high levels of Nos3 (FIGS. 21A-21D). Hematopoietic clusters and adjacent endothelium on the ventral wall of the aorta expressed Nos3 at a lower level; this expression pattern was reminiscent of the embryonic HSC markers such as runx1 and c-kit (North et al., 2002). Fluorescence-activated cell sorting (FACS)-based coexpression analysis confirmed that the majority of e11.5 ckit^{hi}CD34^{med}CD45^{med}VE-cadherin^{med}AGM HSCs (E.D.) were Nos3^{med} (FIG. 21E). Transplantation of Nos3 AGM subfractions into irradiated adult recipients demonstrated that LTR-HSCs are enriched within the Nos3^{med} population (FIG. 21F).

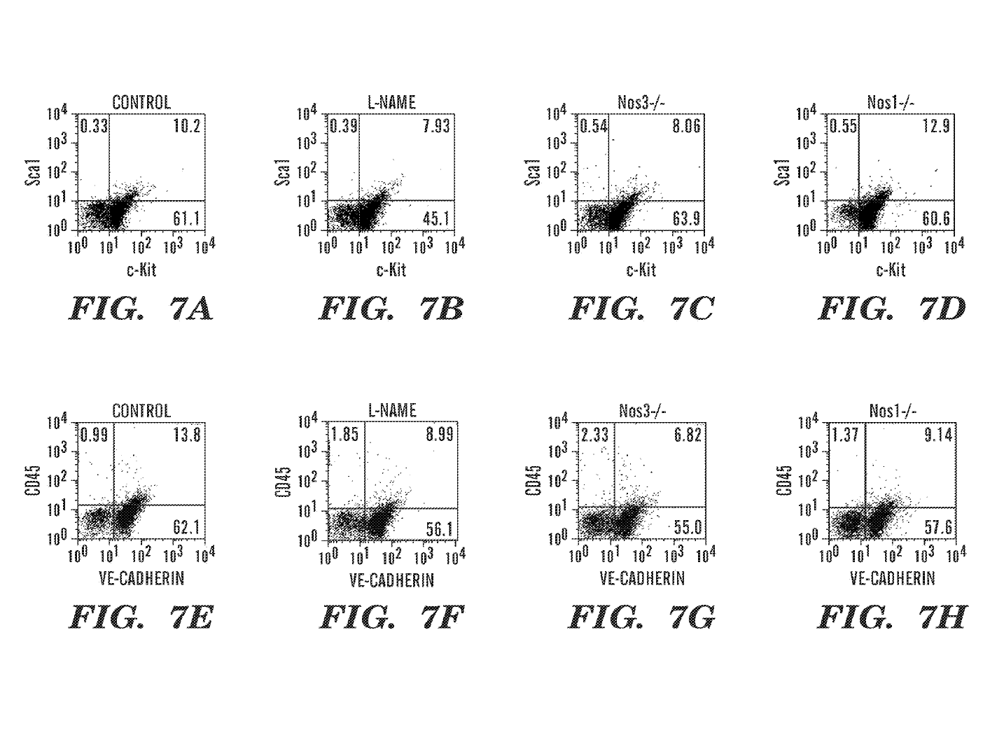

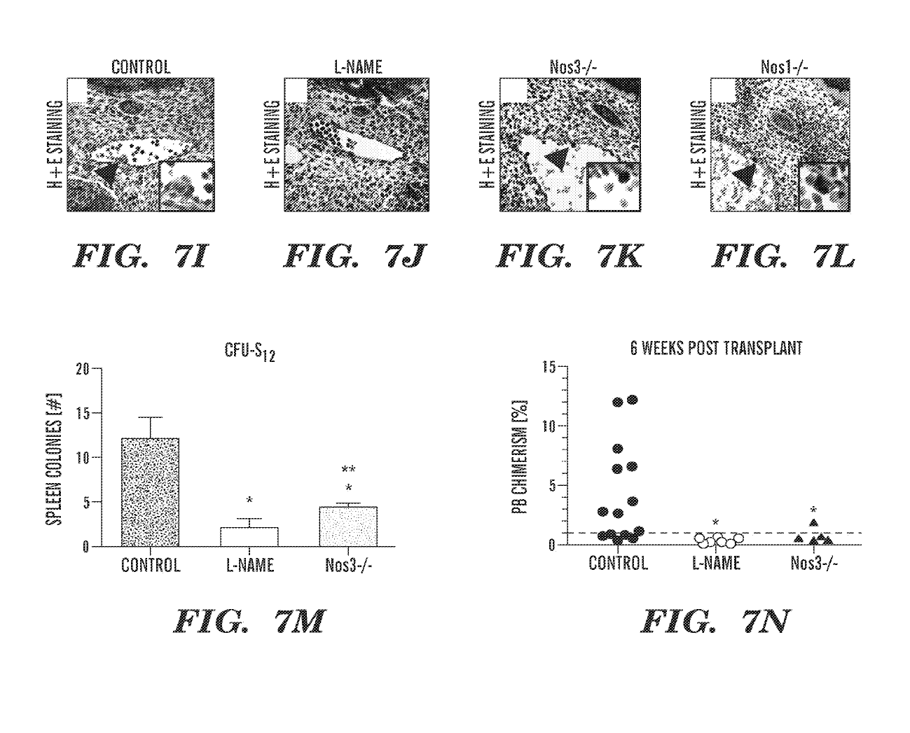

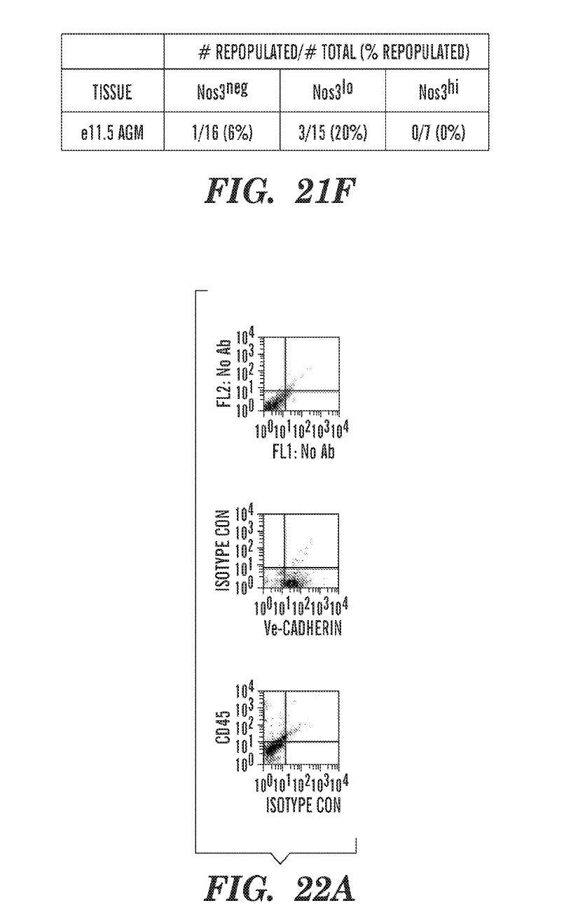

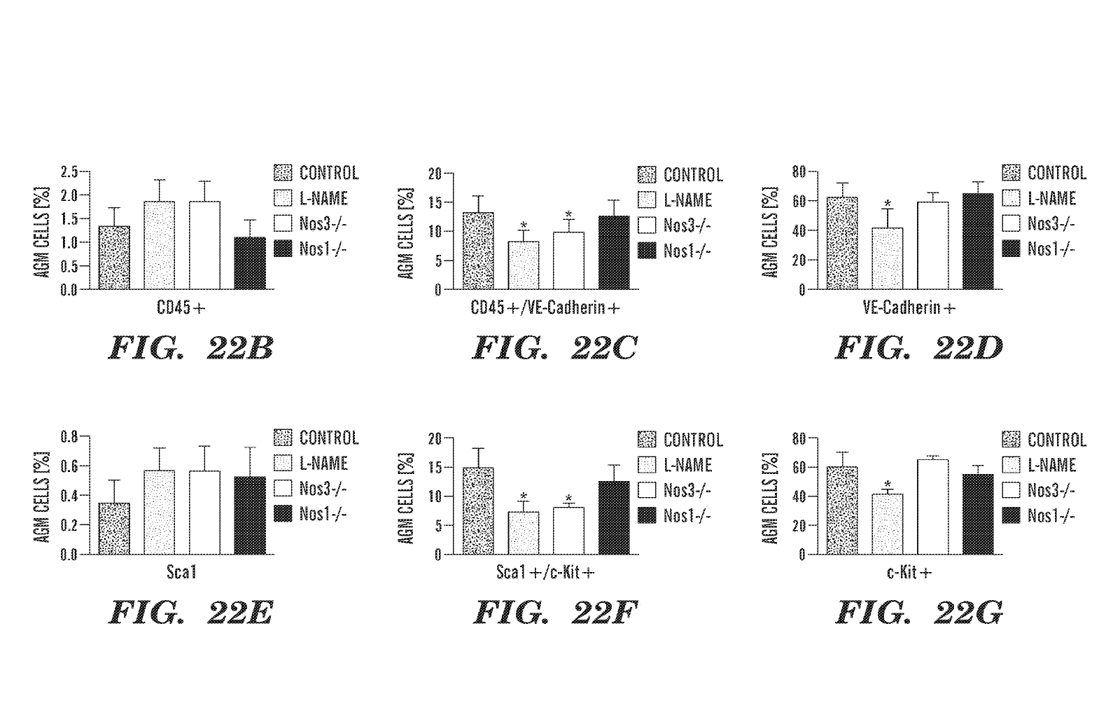

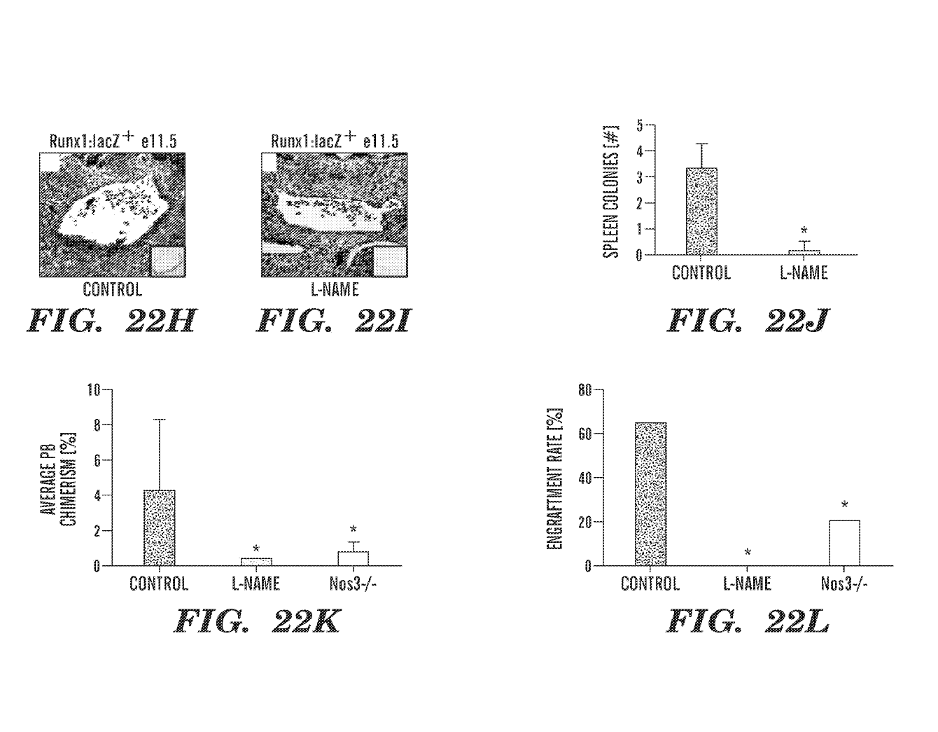

To demonstrate a conserved functional requirement for NO signaling in HSC/progenitor formation, pregnant mice were exposed to L-NAME (2.5 mg/kg intraperitoneally) or vehicle control and compared effects on the AGM HSC and progenitor populations at e11.5. NO inhibition produces implantation defects in early pregnancy (Duran-Reyes et al., 65 Life Sci. 2259-68 (1999)), and can alter yolk sac angiogenesis (Nath et al., 131 Devel. 2485-96 (2004)); interestingly, Nos3 deficiency caused significant lethality from e8.5 to 13.5 during the time when definitive HSCs are formed (Pallares et al., 136 Repro. 573-79 (2008)). L-NAME treatment at e8.5 produced severely delayed embryos that lacked the majority of both extra- and intra-embryonic blood vessels. L-NAME treatment at e9.5 and e10.5 at prevented gross morphological abnormalities of the yolk sac, placenta, or embryo. Histological analysis of the AGM region revealed that L-NAME caused the disappearance of hemogenic endothelial clusters, which was confirmed by phenotypic FACS analysis (FIGS. 7A-7L and 22A-22G). Similarly, analysis of e11.5 Nos3^{-/-} embryos revealed a significant decrease in the AGM sca1+/ckit+ and CD45+/VE-Cadherin+ populations, which was confirmed histologically; Nos3^{-/-} embryos displayed a reduction in the number and size of the hematopoietic clusters (FIG. 7K). HSC induction was grossly normal in Nos1^{-/-} animals (FIG. 7L).

The effects on HSC function were examined by transplantation studies using single-cell suspensions of subdissected AGM tissue from WT, L-NAME-exposed, and Nos3^{-/-} embryos at e11.5. Progenitor activity, as measured by spleen colony formation at days eight and twelve after transplantation, was diminished in L-NAME-exposed (FIG. S15J, p<0.001) and Nos3^{-/-} (FIG. 7M, p<0.001) embryos. Multilineage repopulation after six weeks revealed significantly diminished peripheral blood (PB) chimerism (FIGS. 7N and 22K, p>0.05) and engraftment rates >1% (FIG. 22L) for recipients of both L-NAME-exposed and Nos3^{-/-} AGM cells. These results indicate a conserved role for NO signaling in the regulation of hematopoietic stem/progenitor formation and function during embryonic development. Although at reduced numbers, Nos3^{-/-} embryos develop to adulthood and lack significant steady-state peripheral blood abnormalities; these data suggest that while impaired initially, some functional HSCs do arise in Nos3^{-/-} embryos. Interestingly, although the Nos3^{-/-} animals exhibit some residual HSC production, L-NAME-exposed embryos do not possess AGM HSCs, implying that functional redundancy with other Nos family members must occur.

The purpose of a beating heart and circulation at embryonic stages where diffusion is still sufficient for oxygenation of developing tissue has long been a source for speculation (Burggren, 77 Physiol. Biochecm. Zool. 333-45 (2004); Pelster & Burggren 79 Circ. Res. 358-62 (1996)). Through a chemical screen in Zebrafish, small molecules that regulate vascular dynamics were found to influence HSC development; intriguingly, changes in HSC formation were coupled to blood flow and NO production. The data presented herein imply that circulation itself, through NO induction, signals the onset of definitive hematopoiesis, thereby ensuring proper timing of blood cell development to support additional hematopoietic requirements during accelerated growth in fetal/larval stages. Significantly, the enhancing role of NO in HSC induction is conserved from fish to mammals.

NO production can be induced by sheer stress and alterations in blood flow (Fukumura et al., 2001). The coincident timing of HSC induction with the achievement of vigorous pulsatile flow implies that the latter may serve as the physiologic inductive signal for NO in the AGM. Pulsatile flow achieved by a regular heartbeat has been shown to trigger NO production in the endothelium (White & Frangos, 362 Philos. Trans. R. Soc. Lond. B Biol. Sci. 1459-67 (2007)). The data from the silent heart embryos—as well as observations in Ncx1^{-/-} mice (Lux et al., 111 Blood 3435-38 (2008); Rhodes et al., 2 Cell Stem Cell 252-63 (2008)), which also fail to establish circulation due to heart-specific defects, indicate that in the absence of flow there are alterations in specification, budding, and shedding of HSCs from endothelial hematopoietic lusters.

Studies may further decipher the correlation between flow rate and total AGM HSC number; MO knock down of tnnt2 (sih) (Bertrand et al., 135 Devel. 1853-62 (2008); Murayama et al., 25 Immunity 963-75 (2006); Jin et al., 136 Devel. 647-54 (2009)); and analysis of incompletely penetrant sih mutants with occasional heartbeats that show less-severe reductions in HSC number, implying that small bursts of NO production may be sufficient to trigger HSC induction. As NO can regulate endothelial cell movement and processes resembling HSC budding, such as podokinesis, by altering cell-cell adhesions and actin conformation (Noiri et al., 274 Am. J. Physiol. C236-44 (1998)), it could directly control the formation and stability of hematopoietic clusters once flow is established. This conjecture is confirmed herein: there is a cell-autonomous role of NO signaling during hematopoietic development, where the hemogenic endothelial population must be capable of NO production to support subsequent HSC formation in the AGM.

NO may additionally function to establish the AGM vascular niche prior to HSC formation; the data showing significant alterations in ephrinB2 staining in the absence of flow support the concept that flow itself plays a role in maintaining vascular identity. NO is a well-characterized regulator of angiogenesis and is required for murine yolk sac vasculogenesis (Nath et al., 2004). Prior studies in the Zebrafish embryo showed that chemical inhibition of NO production/signaling by L-NAME or ODQ during somitogenesis produces vascular abnormalities (Pyriochou et al., 319 J. Pharmacol. Exp. Ther. 663-71 (2006)). Because definitive HSCs are formed within the major embryonic arteries (de Bruijn et al., 19 EMBO J. 2465-74 (2000)), any alterations in NO signaling and subsequent vessel development would negatively impact HSC number. As ephrinB2 and arterial identity are established by notch signaling (Lawson et al., 128 Devel. 3675-83 (2001), the interaction of the notch and NO pathways may be relevant for HSC formation. NO may initiate arterial specification early during development and may maintain arterial identity once flow is established. This is in agreement with reports that arterialization is an ongoing and flow-dependent process, influenced by NO (Teichert et al., 103 Circ. Res. 24-33 (2008)). Similarly, vascular endothelial growth factor (VEGF), a potent vascular mitogen regulated by both notch and wnt, is a well-characterized inducer of NO production (Fukumura et al., 98 P.N.A.S. USA 2604-09 (2001)). In the dorsal aorta, VEGF may increase NO production and signaling to cause the vascular remodeling required for the production of the hematopoietic clusters.

The data presented herein demonstrate both a requirement for and enhancing response to NO signaling for AGM HSC development. Several nos isoforms have been identified in Zebrafish: nos1, which is expressed in developing neural tissues as well as the gut, kidney, and major vessels (Holmqvist et al., 207 J. Exp. Biol. 923-35 (2004); Poon et al., 2003), and two isoforms of nos2. Although genomic evidence for the presence in Zebrafish of nos3 is lacking (Pelster et al., 2005), immunoreactivity to eNos antibodies suggests the conservation of the functional epitope (Fritsche et al., 2000). As NO-mediated vascular reactivity is clearly present in fish and nos1 and nos3 are highly related at both the sequence and structural levels, nos1 likely assumes the role of vascular NO production in fish. Nos1 is genetically complex with individual splice forms showing tissue-specific expression, and it is likely that one form of nnos acts enos-like in zebrafish. In support of this hypothesis, microarray analysis demonstrated nos1 expression in both CD41+HSCs and the vascular niche.

In the murine AGM, phenotypic and histological analysis showed that Nos3 (eNos) is expressed in HSCs and required for stem cell function. Conversely, Nos1 (nNos) is not essential under normal developmental conditions. Interestingly, Nos3 and Nos1 are both expressed in the fetal liver shortly after AGM HSC formation and could play a role in the developmental regulation and expansion of HSC and progenitor populations (Krasnov et al., 2008). Their coexpression suggests a functional redundancy in mammalian HSCs that could explain the impaired, but present, HSC formation and adult viability of Nos3^{-/-} embryos. Consequently, global NO inhibition by L-NAME had a much more severe effect on HSC formation. It remains to be determined whether differences in hematopoietic development occur in mice in which all Nos isoforms are disrupted.

NO donors positively affect multipotent hematopoietic progenitors in vitro (Michurina et al., 2004); additionally, the ability of stromal cell lines to support stem cell maintenance corresponds with NO production (Krasnov et al., 2008). In contrast, others have shown that NO inhibition enhances HSC engraftment after transplantation (Krasnov et al., 2008; Michurina et al., 2004). Although these studies imply that NO may have a negative effect on adult HSCs, parallel work has shown that NO is induced by ionizing irradiation and that the absence of Nos diminishes superoxide and peroxide damage (Epperly et al., 2007). These data preclude a clear interpretation of transplantation/repopulation studies where the hematopoietic niche is cleared via irradiation. After 5-fluorouracil bone marrow injury, Nos3^{-/-} mice show impaired regeneration, indicating an important role for Nos3 in stem and progenitor cell function in vivo after marrow injury (Aicher et al., 9 Nat. Med. 1370-76 (2003)). Taken together with the results presented here, these studies indicate that the effects of NO may be time- and context-dependent in vivo, and future work may further decipher the role of NO in regulating adult hematopoietic homeostasis and maintaining both the stromal and vascular niche.

The present invention demonstrates that definitive hematopoietic stem cell formation in the developing embryo is dependent on the induction of the heartbeat and establishment of circulation. Two models have been proposed for the relationship of blood formation in murine extraembryonic tissues and the embryo proper: in one model, the stem cells arise independently in discrete locations in the embryo and extraembryonic tissues and subsequently colonize the fetal liver (Dzierzak & Speck, 2008), whereas the other proposes that cells from the extraembryonic tissues traverse circulation to colonize the intraembryonic hematopoietic sites (Palis & Yoder, 29 Exp. Hematol. 917-36 (2001); Rhodes et al., 2008). A recent study using the Ncx1^{-/-} mouse showed that yolk sac hematopoietic progenitors could form in the absence of blood flow, while the appearance of progenitors in the embryo proper was greatly impaired; these data were interpreted to show that it is yolk sac-derived embryonic progenitors that traverse the circulation and seed the fetal liver (Lux et al., 2008). The data presented herein imply that the contemporaneous establishment of circulation and the appearance of HSCs within the embryo proper may not simply reflect the transit of HSCs formed in extraembryonic tissues to colonize the aorta and fetal liver, but rather that the circulation functions directly to provide inductive signals to specific regions of the embryonic vasculature, making it competent to produce HSCs de novo.

The present invention provides for a conserved role for NO in the developing hematopoietic system. NO can function in vessel formation and specification, blood flow regulation, and hematopoietic cluster formation, suggesting that it is required in the vascular niche for HSC production.

The present invention thus provides methods for modulating HSC growth and renewal in vitro, or ex vivo. In one embodiment, the invention provides methods for promoting HSC growth and renewal in a cell population. The method comprises, for example, contacting a nascent stem cell population with at least one HSC modulator. This population may be contained within peripheral blood, cord blood, bone marrow, amniotic fluid, chorionic villa, placenta, or other hematopoietic stem cell niches. Hence, an embodiment of the present invention provides for NO pathway modulators (and associated downstream pathway modulators) that may be used for the induction of HSCs from ESC, induced pluripotent stem cell (iPSC), or AGM cell populations. Previous work (North et al., 2007), has shown that prostaglandins and (conversely) cox2 inhibitors (in the same assay) had effects on embryonic HSC proliferation; and demonstrated effects on mouse ESC differentiation into hematopoietic colony forming units.

In another embodiment, the invention provides methods for inhibiting hematopoietic stem cell growth and renewal in a cell population.

The present invention is based, in part, on the discovery that NO and agents that enhance NO signaling, including agents that increase blood circulation, cause an increase in HSC numbers. Conversely, agents that block NO signaling, including those that decrease blood circulation, decrease HSCs. In that regard, agents affecting NO signaling may be considered HSC modulators. For example, S-nitroso-N-acetyl-penicillamine (SNAP) increases HSC formation; conversely, N-nitro-L-arginine methyl ester (L-NAME) reduces HSC formation. These agents are thus considered HSC modulators.

As used herein, HSC modulators may either promote or inhibit HSC growth and renewal in vitro and ex vivo. HSC modulators influence HSC numbers in a cell population. HSC modulators influence HSC expansion in culture (in vitro), during short term incubation, (ex vivo). HSC modulators that increase HSC numbers include agents that upregulate NO signaling. An increase in HSC numbers can be an increase of about 10%, about 20%, about 30%, about 40%, about 50%, about 60%, about 70%, about 80%, about 90%, about 100%, about 150%, about 200% or more, than the HSC numbers exhibited by in vitro or ex vivo culture prior to treatment.

The HSC modulators of the present invention also include derivatives of HSC modulators. Derivatives, as used herein, include a chemically modified compound wherein the modification is considered routine by the ordinary skilled chemist, such as additional chemical moieties (e.g., an ester or an amide of an acid, protecting groups, such as a benzyl group for an alcohol or thiol, and tert-butoxycarbonyl group for an amine). Derivatives also include radioactively labeled HSC modulators, conjugates of HSC modulators (e.g., biotin or avidin, with enzymes such as horseradish peroxidase and the like, with bioluminescent agents, chemoluminescent agents or fluorescent agents). Additionally, moieties may be added to the HSC modulator or a portion thereof to increase half-life. Derivatives, as used herein, also encompasses analogs, such as a compound that comprises a chemically modified form of a specific compound or class thereof, and that maintains the pharmaceutical and/or pharmacological activities characteristic of said compound or class, are also encompassed in the present invention. Derivatives, as used herein, also encompasses prodrugs of the HSC modulators, which are known to enhance numerous desirable qualities of pharmaceuticals (e.g., solubility, bioavailability, manufacturing, etc.).

Ex vivo administration of HSC modulators can enable significant expansion of hematopoietic stem cells, such that even small amounts of hematopoietic stem cells can be expanded enough for transplantation. Consequently, for example, cord blood stem cell transplantation may now be applied to not only children but also adults. Such stem cells may be collected from sources including, for example, peripheral blood, cord blood, bone marrow, amniotic fluid, or placental blood. Alternatively, the HSC-containing source sample may be harvested and then stored immediately in the presence of a HSC modulator, such as SNAP, and initially incubated (prior to differentiation) in the presence of the HSC modulator before HSC introduction into a subject. Further, by increasing the number of HSC available for transplantation back into the subject or to another subject, potentially reduces the time to engraftment, and consequently decreases in the time during which the subject has insufficient neutrophils and platelets, thus preventing infections, bleeding, or other complications.

In vitro expansion of HSC, according to the present invention, also provides HSC sources for drug screening and further research.

HSC modulators can also be used ex vivo to provide autologous HSCs to a subject. Typically, this involves the steps of harvesting bone marrow stem cells or stem cells in the peripheral circulation; expanding the cell population; and transplanting the expanded harvested stem cells back into the subject.

In addition, the stem cells obtained from harvesting according to method of the present invention described above can be cryopreserved using techniques known in the art for stem cell cryopreservation. Accordingly, using cryopreservation, the stem cells can be maintained such that once it is determined that a subject is in need of stem cell transplantation, the stem cells can be thawed and transplanted back into the subject. As noted previously, the use of one or more HSC modulators, for example SNAP, during cryopreservation techniques may enhance the HSC population.

More specifically, an embodiment of the present invention provides for the enhancement of HSCs collected from cord blood or an equivalent neonatal or fetal stem cell source, which may be cryopreserved, for the therapeutic uses of such stem cells upon thawing. Such blood may be collected by several methods known in the art. For example, because umbilical cord blood is a rich source of HSCs (see Nakahata & Ogawa, 70 J. Clin. Invest. 1324-28 (1982); Prindull et al., 67 Acta. Paediatr. Scand. 413-16 (1978); Tchernia et al., 97(3) J. Lab. Clin. Med. 322-31 (1981)), an excellent source for neonatal blood is the umbilical cord and placenta. The neonatal blood may be obtained by direct drainage from the cord and/or by needle aspiration from the delivered placenta at the root and at distended veins. See, e.g., U.S. Pat. No. 7,160,714; No. 5,114,672; No. 5,004,681; U.S. patent application Ser. No. 10/076,180, Pub. No. 20030032179.

Indeed, umbilical cord blood stem cells have been used to reconstitute hematopoiesis in children with malignant and nonmalignant diseases after treatment with myeloablative doses of chemo-radiotherapy. Sirchia & Rebulla, 84 Haematologica 738-47 (1999). See also Laughlin 27 Bone Marrow Transplant. 1-6 (2001); U.S. Pat. No. 6,852,534. Additionally, it has been reported that stem and progenitor cells in cord blood appear to have a greater proliferative capacity in culture than those in adult bone marrow. Salahuddin et al., 58 Blood 931-38 (1981); Cappellini et al., 57 Brit. J. Haematol. 61-70 (1984).

Alternatively, fetal blood can be taken from the fetal circulation at the placental root with the use of a needle guided by ultrasound (Daffos et al., 153 Am. J. Obstet. Gynecol. 655-60 (1985); Daffos et al., 146 Am. J. Obstet. Gynecol. 985-87 (1983), by placentocentesis (Valenti, 115 Am. J. Obstet. Gynecol. 851-53 (1973); Cao et al., 19 J. Med. Genet. 81-87 (1982)), by fetoscopy (Rodeck, in PRENATAL DIAGNOSIS, (Rodeck & Nicolaides, eds., Royal College of Obstetricians & Gynaecologists, London, 1984)). Indeed, the chorionic villus and amniotic fluid, in addition to cord blood and placenta, are sources of pluripotent fetal stem cells (see WO 2003 042405) that may be treated by the HSC modulators of the present invention.

Various kits and collection devices are known for the collection, processing, and storage of cord blood. See, e.g., U.S. Pat. No. 7,147,626; No. 7,131,958. Collections should be made under sterile conditions, and the blood may be treated with an anticoagulant. Such an anticoagulants include citrate-phosphate-dextrose, acid citrate-dextrose, Alsever's solution (Alsever & Ainslie, 41 N.Y. St. J. Med. 126-35 (1941), DeGowin's Solution (DeGowin et al., 114 JAMA 850-55 (1940)), Edglugate-Mg (Smith et al., 38 J. Thorac. Cardiovasc. Surg. 573-85 (1959)), Rous-Turner Solution (Rous & Turner, 23 J. Exp. Med. 219-37 (1916)), other glucose mixtures, heparin, or ethyl biscoumacetate. See Hum, STORAGE OF BLOOD 26-160 (Acad. Press, NY, 1968).

Various procedures are known in the art and can be used to enrich collected cord blood for HSCs. These include but are not limited to equilibrium density centrifugation, velocity sedimentation at unit gravity, immune rosetting and immune adherence, counterflow centrifugal elutriation, T-lymphocyte depletion, and fluorescence-activated cell sorting, alone or in combination. See, e.g., U.S. Pat. No. 5,004,681.

Typically, collected blood is prepared for cryogenic storage by addition of cryoprotective agents such as DMSO (Lovelock & Bishop, 183 Nature 1394-95 (1959); Ashwood-Smith 190 Nature 1204-05 (1961)), glycerol, polyvinylpyrrolidine (Rinfret, 85 Ann. N.Y. Acad. Sci. 576-94 (1960)), polyethylene glycol (Sloviter & Ravdin, 196 Nature 899-900 (1962)), albumin, dextran, sucrose, ethylene glycol, i-erythritol, D-ribitol, D-mannitol (Rowe, 3(1) Cryobiology 12-18 (1966)), D-sorbitol, i-inositol, D-lactose, choline chloride (Bender et al., 15 J. Appl. Physiol. 520-24 (1960)), amino acids (Phan & Bender, 20 Exp. Cell Res. 651-54 (1960)), methanol, acetamide, glycerol monoacetate (Lovelock, 56 Biochem. J. 265-70 (1954)), and inorganic salts (Phan & Bender, 104 Proc. Soc. Exp. Biol. Med. (1960)). Addition of plasma (e.g., to a concentration of 20%-25%) may augment the protective effect of DMSO.

Collected blood should be cooled at a controlled rate for cryogenic storage. Different cryoprotective agents and different cell types have different optimal cooling rates. See e.g., Rapatz, 5 Cryobiology 18-25 (1968), Rowe & Rinfret, 20 Blood 636-37 (1962); Rowe, 3 Cryobiology 12-18 (1966); Lewis et al., 7 Transfusion 17-32 (1967); Mazur, 168 Science 939-49 (1970). Considerations and procedures for the manipulation, cryopreservation, and long-term storage of HSC sources are known in the art. See e.g., U.S. Pat. No. 4,199,022; No. 3,753,357; No. 4,559,298; No. 5,004,681. There are also various devices with associated protocols for the storage of blood. U.S. Pat. No. 6,226,997; No. 7,179,643

Considerations in the thawing and reconstitution of HSC sources are also known in the art. U.S. Pat. No. 7,179,643; No. 5,004,681. The HSC source blood may also be treated to prevent clumping (see Spitzer, 45 Cancer 3075-85 (1980); Stiff et al., 20 Cryobiology 17-24 (1983), and to remove toxic cryoprotective agents (U.S. Pat. No. 5,004,681). Further, there are various approaches to determining an engrafting cell dose of HSC transplant units. See U.S. Pat. No. 6,852,534; Kuchler, in BIOCHEM. METHS CELL CULTURE & VIROLOGY 18-19 (Dowden, Hutchinson & Ross, Strodsburg, Pa., 1964); 10 METHS. MED. RES. 39-47 (Eisen, et al., eds., Year Book Med. Pub., Inc., Chicago, Ill., 1964).

Thus, not being limited to any particular collection, treatment, or storage protocols, an embodiment of the present invention provides for the addition of an HSC modulator, such as SNAP, to the neonatal blood. This may be done at collection time, or at the time of preparation for storage, or upon thawing and before infusion.

For example, stem cells isolated from a subject, e.g., with or without prior treatment of the subject with HSC modulators, may be incubated in the presence of HSC modulators, e.g., HSC modulators such as SNAP to expand the number of HSCs. Expanded HSCs may be subsequently reintroduced into the subject from which they were obtained or may be introduced into another subject.

The HSC modulators, including SNAP and the compounds disclosed herein, can thus be used for, inter alia: reducing the time to engraftment following reinfusion of stem cells in a subject; reducing the incidence of delayed primary engraftment; reducing the incidence of secondary failure of platelet production; and reducing the time of platelet and/or neutrophil recovery following reinfusion of stem cells in a subject. These methods typically include the steps of harvesting the bone marrow stem cells or the stem cells in the peripheral circulation, expanding the stem cells in vitro by exposing the cells to an HSC modulator (e.g., SNAP), and then transplanting the expanded stem cells back into the subject at the appropriate time, as determined by the particular needs of the subject.

The method of the invention may also be used to ex vivo increase the number of stem cells from a donor subject (including bone marrow cells or cord blood cells), whose cells are then used for rescue of a recipient subject who has received bone marrow ablating chemotherapy or irradiation therapy. As used herein, a subject includes anyone who is a candidate for autologous stem cell or bone marrow transplantation during the course of treatment for malignant disease or as a component of gene therapy. Subjects may have undergone irradiation therapy, for example, as a treatment for malignancy of cell type other than hematopoietic. Subjects may be suffering from anemia, e.g., sickle cell anemia, thalessemia, aplastic anemia, or other deficiency of HSC derivatives.

The method of the invention thus provides the following benefits: (1) Allows transplantation to proceed in patients who would not otherwise be considered as candidates because of the unacceptably high risk of failed engraftment; (2) Reduces the number of aphereses required to generate a minimum acceptable harvest; (3) Reduces the incidence of primary and secondary failure of engraftment by increasing the number HSCs available for transplantation; and (4) Reduces the time required for primary engraftment by increasing the number of committed precursors of the important hemopoietic lineages.

Various kits and collection devices are known for the collection, processing, and storage of source cells are known in the art. The modulators of the present invention may be introduced to the cells in the collection, processing, and/or storage. Thus, not being limited to any particular collection, treatment, or storage protocols, an embodiment of the present invention provides for the addition of a modulator, such as, for example, SNAP or its analogs, to a tissue sample. This may be done at collection time, or at the time of preparation for storage, or upon thawing and before implantation.

Several embodiments will now be described further by non-limiting examples.

### EXAMPLES

### Example 1

### Zebrafish

Husbandry Zebrafish were maintained according to Institutional Animal Care and Use Committee protocols. fli:GFP, hs:gal4; uas:NICD, wnt8:GFP, dkk1:GFP transgenic and sih and mib mutant fish were described previously (Burns et al., 2005; Goessling et al., 2008b; Itoh et al., 4 Devel. Cell 67-82 (2003); Lawson & Weinstein, 248 Devel. Bio. 307-18 (2002); Sehnert et al., 2002). Embryonic heat shock was conducted as described (Goessling et al., 2008b).

In Situ Hybridizataion: Paraformaldehyde-fixed embryos were processed for in situ hybridization according to standard zebrafish protocols. Such protocols are available on-line through the The Zebrafish Model Organism Database. The following RNA probes were used: runx1, cmyb, flk1, ephrinB2, flt4, globin, mpo, mhc, and foxa3. Changes in expression compared to WT controls are reported as the number altered/number scored per genotype/treatment (North et al., 2007); a minimum of three independent experiments was conducted per analysis.

Chemical Exposure: Zebrafish embryos were exposed to chemicals at the doses indicated; dimethyl sulfoxide (DMSO) carrier content was 0.1%. For evaluation of HSC development, exposure ranged from early somitogenesis (5+ somites) until 36 hpf, unless otherwise noted.

Blastula Transplantation: cmyb: GFP embryos were injected with nos1 or control MO at the one-cell stage. At the blastula stage, 100 cells were removed from the donor embryo and transplanted into stage-matched recipients. Embryos were analyzed by confocal microscopy at 36 hpf.

Morpholino injection: Morpholino antisense oligonucleotides (MO; GENETOOLS, LLC, Philomath, Oreg.) designed against the ATG and exonl splice sites of nos1 (5′-ACGCTGGGCTCTGATTCCTGCATTG [SEQ. ID NO:1]; 5′-TTAATGACATCCCTCACCTCTCCAC [SEQ ID NO:2), nos2 (5′-AGTGGTTTGTGCTTGTCTTCCCATC [SEQ ID NO:3]; 5′-ATGCATTAGTACCTTTGATTGCACA [SEQ ID NO:4]), and mismatched controls were injected into one-cell-stage embryos.

Confocal Microscopy: Fluorescent reporter embryos were exposed to blood flow modulators (10 μM, unless otherwise noted) as indicated, live embedded in 1% agarose, and imaged with a Zeiss LSM510 Meta confocal microscope at 36 hpf (North et al., 2007) or a Perkin Elmer UltaVIEW VoX spinning disk confocal microscope.

### Example 2

### Mice

Embryos were generated from C57B1/6, Runx1:lacZ (North et al., 1999), Nos3:GFP transgenic (van Haperen et al., 163 Am. J. Pathol. 1677-86 (2003)), Nos1^{-/-}, and Nos3^{-/-} mice. Vaginal plug identification was considered e0.5. Animals were handled according to institutional guidelines.

Murine AGM Histology: At e11.5 after timed mating, embryos dissected from the uterus and processed for histological evaluation. Paraffin serial sections were stained with hematoxylin and eosin; cryosections were assessed by fluorescence microscopy for GFP. X-Gal staining was performed as indicated.

Nos3:GFP AGM Transplantation: Transgenic AGM cells were sorted into Nos3:GFP fractions. AGM (one embryo equivalent) cell suspensions were injected into irradiated (9 Gy) FVB recipient mice with adult spleen carrier cells (2×10⁵ per recipient). Recipient peripheral blood was analyzed at 4 months after transplantation for donor-derived cells by DNA PCR for GFP (donor marker) and myogenin (normalization control). Recipients with >10% donor-marked cells were considered positive.

AGM Transplantation and Progenitor and LTR HSC Analysis: AGM transplantations were performed with the CD45.1/45.2 allelic system. Pregnant C57B1/6 females were injected with DMSO or L-NAME (2.5 mg/kg) intraperitoneally on e9.5 and e10.5. WT, L-NAME, Nos1^{-/-}, and Nos3^{-/-} AGM regions were dissected and disaggregated at e11.5 then injected into 8-week-old C57B1/6 sublethally irradiated recipients. For CFUS8 and 12 analyses, spleens were dissected, weighed, and fixed with Bouin's solution, and hematopoietic colonies were counted. For long-term transplants, PB obtained from recipient mice at six weeks was analyzed for donor chimerism and multilineage engraftment by FACS.

Nos3:GFP AGM FACS Analysis: Embryos (e11.5) from Nos3: GFP transgenic animals were isolated, and AGM tissue was dissected and disaggregated. Flow cytometric analysis was performed for Nos3:GFP, VE-Cadherin, CD34, Sca-1, c-kit, and CD45 (BD Biosciences Pharmingen, San Jose, Calif.).

### Example 3

### qPCR

qPCR was performed on cDNA obtained from whole embryos at 36 hpf (n=20/variable; primers listed in Table 1 (below) as previously described (North et al., 2007), with SYBR Green Supermix on the iQ5 Multicolor RTPCR Detection System (BioRad).

TABLE 1
PCR primer sequences

| | | |
|---|---|---|
| beta actin F | 5′-GCTGTTTTCCCCTCCATTGTT | SEQ ID |
| | | NO: 5 |
| beta actin R | 5′-TCCCATGCCAACCATCACT | SEQ ID |
| | | NO: 6 |
| runx1 F | 5′-CGTCTTCACAAACCCTCCTCAA | SEQ ID |
| | | NO: 7 |
| runx1 R | 5′-CGTTTACTGCTTCATCCGGCT | SEQ ID |
| | | NO: 8 |
| cmyb F | 5′-TGATGCTTCCCAACACAGAG | SEQ ID |
| | | NO: 9 |
| cmyb R | 5′-TTCAGAGGGAATCGTCTGCT | SEQ ID |
| | | NO: 10 |
| flk1 F | 5′-CGAACGTGAAGTGACATACGG | SEQ ID |
| | | NO: 11 |
| flk1 R | 5′-CCCTCTACCAAACCATGTGAAA | SEQ ID |
| | | NO: 12 |
| ephrin B2 F | 5′-CAAGGACAGCAAATCGAATG | SEQ ID |
| | | NO: 13 |
| ephrin B2 R | 5′-TGAGCCAATGACTGATGAGG | SEQ ID |
| | | NO: 14 |
| nos1 F | 5′-CTCCATTCAGAGCCTTCTGG | SEQ ID |
| | | NO: 15 |
| nos1 R | 5′-CCGACAACCAAACACCAAG | SEQ ID |
| | | NO: 16 |
| nos2 F | 5′-AGGCACTCGTGGCTATCAAT | SEQ ID |
| | | NO: 17 |
| nos2 R | 5′-ATGCTGCATGAAGGACTCG | SEQ ID |
| | | NO: 18 |
| nos1 splice | 5′-TGGGGTGGAGGATAACAATG | SEQ ID |
| junct F | | NO: 19 |
| nos1 splice | 5′-ACAGCCTTGGTAGGAGAAACTC | SEQ ID |
| junct R | | NO: 20 |

1. A method for promoting hematopoietic stem cell (HSC) growth comprising contacting embryonic stem cells (ESC), induced pluripotent stem cells (iPSC), aorta-gonads-mesonephros (AGM) cells or HSC with at least one HSC growth modulator that up-regulates the nitric oxide (NO) signaling pathway.

2. The method of claim 1 wherein said HSC modulator is NO or S-nitroso-N-acetyl-penicillamine (SNAP).

3. The method of claim 1, wherein said HSC modulator is Doxasozin, Metoprolol, Nifedipine, Digoxin, NO, SNAP, L-ARG, Todralazine, Sodium Nitroprusside, Atenolol, Pronethalol, Pindolol, Fendiline, Nicardipine, Strophanthidin, Lanatoside, Peruvoside, Histamine, Hydralazine, or Todralazine.

4. A method for promoting HSC expansion comprising incubating a cell population comprising at least one iPSC, ESC, AGM HSC or HSC in the presence of at least one HSC modulator selected from the group consisting of Doxasozin, Metoprolol, Nifedipine, Digoxin, NO, SNAP, L-ARG, Todralazine, Sodium Nitroprusside, Atenolol, Pronethalol, Pindolol, Fendiline, Nicardipine, Strophanthidin, Lanatoside, Peruvoside, Histamine, Hydralazine, and Todralazine.

5-8. (canceled)

9. A method for inhibiting HSC growth in a cell population, comprising contacting said cell population with at least one HSC modulator and a pharmaceutically acceptable carrier, wherein the HSC modulator down-regulates the NO signaling pathway and is selected from the group consisting of Ergotamine, Epinephrine, BayK8644, L-NAME, Chrysin, Enalapril, Ephedrine, Methoxamine, Mephentermine, Propranolol, Nerifolin, Proadifen, Ambroxol, and Captopril.

10. A method for increasing the number of hematopoietic stem cells (HSC) in a subject, comprising administering at least one HSC modulator that up-regulates the nitric oxide (NO) signaling pathway and a pharmaceutically acceptable carrier to the subject.

11. The method of claim 10, wherein the subject is human.

12. The method of claim 10, wherein the subject has a decreased blood cell level or is at risk for developing a decreased blood cell level as compared to a control blood cell level.

13. The method of claim 10, wherein the subject has anemia or blood loss.

14. The method of claim 10, wherein the subject is a bone marrow donor.

15. The method of claim 10, wherein the subject has depleted bone marrow.

16. The method of claim 10, wherein said HSC modulator is NO or S-nitroso-N-acetyl-penicillamine (SNAP).

17. The method of claim 10, wherein said HSC modulator is Doxasozin, Metoprolol, Nifedipine, Digoxin, NO, SNAP, L-ARG, Todralazine, Sodium Nitroprusside, Atenolol, Pronethalol, Pindolol, Fendiline, Nicardipine, Strophanthidin, Lanatoside, Peruvoside, Histamine, Hydralazine, or Todralazine.

18. (canceled)

19. A method for inhibiting HSC growth in a subject, comprising administering at least one HSC modulator and a pharmaceutically acceptable carrier, wherein the HSC modulator down-regulates the NO signaling pathway and is selected from the group consisting of Ergotamine, Epinephrine, BayK8644, L-NAME, Chrysin, Enalapril, Ephedrine, Methoxamine, Mephentermine, Propranolol, Nerifolin, Proadifen, Ambroxol, and Captopril.
